(19) United States
(12) Patent Application Publication
(10) Pub. No.: US 2026/0308589 A1
(43) Pub. Date: Oct. 8, 2026

(54) IDENTIFICATION OF HOST FACTORS AND COMPOUNDS TO BOOST VIRAL TRANSDUCTION

(71) Applicant: UNIVERSITY OF MASSACHUSETTS, Westborough, MA (US)

(72) Inventors: Jun Xie, Shrewsbury, MA (US); Guangping Gao, Westborough, MA (US); Jisun Lee, Shrewsbury, MA (US)

(21) Appl. No.: 19/483,101

(22) PCT Filed: May 10, 2024
(86) PCT No.: PCT/US2024/028726

§ 371 Date: Nov. 10, 2025

Related U.S. Application Data
(60) Provisional application No. 63/501,441, filed on May 11, 2023.

(51) Int. Cl. C12N 15/113; A61K 31/4355; A61K 31/496; C12N 15/86

(52) U.S. Cl. CPC C12N 15/113; A61K 31/4355; A61K 31/496; C12N 15/86; C12N 2310/141; C12N 2750/14143

(57) ABSTRACT

Provided herein are a) pharmaceutical compositions comprising a vector, a host protein inhibitor, and a pharmaceutically acceptable carrier; b) methods of delivering a transgene to a cell in a subject, said method comprising administering to the subject a pharmaceutical composition comprising a vector and a host protein inhibitor; c) methods of increasing transduction efficiency of a vector to a cell, said method comprising contacting the cell with a host protein inhibitor; and d) methods of treating a disease in a subject, wherein the method comprises administering to the subject in need thereof a pharmaceutical composition comprising a vector and a host protein inhibitor.

### RELATED APPLICATION

This application claims the benefit of U.S. Provisional Application No. 63/501,441, filed on May 11, 2023. The entire teachings of the above application are incorporated herein by reference.

### BACKGROUND

Adeno-associated virus (AAV), a member of the Parvovirus family, is a small nonenveloped, icosahedral virus with single-stranded linear DNA genomes of 4.7 kilobases (kb) to 6 kb. AAV is assigned to the genus, Dependovirus, because the virus was discovered as a contaminant in purified adenovirus stocks. AAV's life cycle includes a latent phase at which AAV genomes, after infection, are site specifically integrated into host chromosomes and an infectious phase in which, following either adenovirus or herpes simplex virus infection, the integrated genomes are subsequently rescued, replicated, and packaged into infectious viruses. The properties of non-pathogenicity, broad host range of infectivity, including non-dividing cells, and potential site-specific chromosomal integration make AAV an attractive tool for gene transfer.

### SUMMARY

Disclosed herein is a pharmaceutical composition comprising a vector, a host protein inhibitor or a stereoisomer, tautomer or salt thereof, and a pharmaceutically acceptable carrier, wherein the vector comprises a transgene operably linked to regulatory sequences which direct expression of a product from the transgene in a cell. In some embodiments, the host protein is eIF4A1, eIF4A2, eIF4A3, RAP1A, NIFK, SART1, PLS1, or SF3B6.

Also disclosed herein is a method of delivering a transgene to a cell in a subject, said method comprising administering to the subject a pharmaceutical composition comprising a vector and a host protein inhibitor or a stereoisomer, tautomer or salt thereof, wherein the vector comprises the transgene operably linked to regulatory sequences which direct expression of a product from the transgene in the cell. In some embodiments, the host protein is eIF4A1, eIF4A2, eIF4A3, RAP1A, NIFK, SART1, PLS1, or SF3B6.

A method of increasing transduction efficiency of a vector to a cell is disclosed herein, said method comprising contacting the cell with a host protein inhibitor or a stereoisomer, tautomer or salt thereof, wherein the vector comprises a transgene operably linked to regulatory sequences which direct expression of a product from the transgene in the cell. In some embodiments, the host protein is eIF4A1, eIF4A2, eIF4A3, RAP1A, NIFK, SART1, PLS1, or SF3B6.

A method of treating a disease in a subject is also disclosed herein, wherein the method comprises administering to the subject in need thereof a pharmaceutical composition comprising a vector and a host protein inhibitor or a stereoisomer, tautomer or salt thereof, wherein the vector comprises a transgene operably linked to regulatory sequences which direct expression of a product from the transgene in a cell. In some embodiments, the host protein is eIF4A1, eIF4A2, eIF4A3, RAP1A, NIFK, SART1, PLS1, or SF3B6.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, including those illustrated in the drawings interspersed herein. The drawings are not necessarily to scale, emphasis instead being placed upon illustrating embodiments.

FIG. 1A: Scheme for identifying host proteins involved in AAV transduction by proximity-dependent labeling using proximity-dependent biotin identification (BioID).

FIG. 1B: Identification of biotintylated and non-biotintylated viral proteins 1, 2, and 3.

FIG. 2A: Validation of biotin ligase activity.

FIG. 2B: Western blot of in vitro biotin reaction products.

FIG. 3: Gene Ontology Enrichment Analysis with 77 host protein candidates.

FIG. 4: Scheme of transduction efficiency test. Cells are transfected with siRNA for 48 hours, followed by infection with ssAAV2-luciferase for 24 hours. Luminescence intensity was then measured.

FIG. 5: Transduction efficiency of ssAAV2-Luciferase in HEK 293 cells with various host protein knock-downs and without any host protein knock-down (24 hours post-infection). ID codes-13: EIF4A3, 17: RAP1A, 18: NIFK, 31: SART1, 10: PLS1, 19: SF3B6, 21: RUVBL1, 6: RAB11FIP2, 9: DCXR, 12: PSMD13, 27:TBL2.

FIG. 6: Transduction efficiency of ssAAV2-Luciferase in Hela cells with various host protein knock-downs and without any host protein knock-down (24 hours post-infection).

FIG. 7: Scheme of transduction efficiency test for AAV serotypes. Cells are transfected with siRNA for 48 hours, followed by infection with ssAAV-luciferase for 24 hours. Luminescence intensity was then measured.

FIG. 8: Transduction efficiency of ssAAV1-Luciferase in Hela cells with various host protein knock-downs and without any host protein knock-down.

FIG. 9: Transduction efficiency of ssAAV3b-Luciferase in HeLa cells with various host protein knock-downs and without any host protein knock-down.

FIG. 10: Transduction efficiency of ssAAV5-Luciferase in HeLa cells with various host protein knock-downs and without any host protein knock-down.

FIG. 11: Transduction efficiency of ssAAV9-Luciferase in Hela cells with various host protein knock-downs and without any host protein knock-down.

FIG. 12A: Scheme of host protein knockdown experiment for investigating AAV attachment.

FIG. 12B: Bound ssAAV2-luciferase virions in HeLa cells (with knockdown of eukaryotic initiation factor 4A-III (eIF4A3)) were quantified by qPCR for luciferase gene levels. Multiplicity of Infection (MOI) is 10,000.

FIG. 13A: Scheme of host protein knockdown experiment for investigating AAV internalization.

FIG. 13B: Internalized ssAAV2-luciferase virions in HeLa cells (with knockdown of eIF4A3) were quantified by qPCR for luciferase gene levels. Multiplicity of Infection (MOI) is 10,000.

FIG. 14A: Scheme of host protein knockdown experiment for investigating nuclear entry of AAV.

FIG. 14B: Fold change of luciferase activity in cytoplasmic and nuclear fractions from HeLa cells with and without knockdown of eIF4A3 after 4 hours from infection phase. Multiplicity of Infection (MOI) is 10,000.

FIG. 14C: Fold change of luciferase activity in cytoplasmic and nuclear fractions from HeLa cells with and without knockdown of eIF4A3 after 6 hours from infection phase. Multiplicity of Infection (MOI) is 10,000.

FIG. 15A: Scheme of host protein knockdown experiment for investigating AAV uncoating.

FIG. 15B: Fold change of luciferase activity in nuclear fractions from HeLa cells with and without knockdown of eIF4A3 after 4 hours and 6 hours from infection phase. Multiplicity of Infection (MOI) is 10,000.

FIG. 16A: Scheme of host protein knockdown experiment for investigating conversion from single strand DNA to double stranded DNA.

FIG. 16B: Fold change of gaussia luciferase activity from HeLa cells with and without knockdown of eIF4A3 at various concentrations of siRNA (nM). Multiplicity of Infection (MOI) is 10,000.

FIG. 17A: Scheme of host protein knockdown experiment for investigating AAV genome transcription.

FIG. 17B: Fold change of luciferase mRNA transcript levels with and without knockdown of eIF4A3. Multiplicity of Infection (MOI) is 10,000.

FIG. 18A: Scheme of AAV transduction experiment for investigating overexpression of ORF clones.

FIG. 18B: Western blot analysis of eIF4A3 with respect to beta tubulin for overexpression of ORF clones.

FIG. 18C: Fold change of transduction efficiency with overexpression of ORF clones normalized by empty vector (pcDNA3.1). Multiplicity of Infection (MOI) is 10,000.

FIG. 19A: Scheme of experiment investigating eIF4A inhibition on cell viability.

FIG. 19B: Cell viability after pre-treatment with eIF4A3-IN-1 for 6 hours.

FIG. 19C: Cell viability after pre-treatment with eIF4A3-IN-1 for 6 hours, followed by infection with ssAAV-luciferase for 24 hours.

FIG. 20A: Scheme of experiment investigating eIF4A inhibition on AAV transduction.

FIG. 20B: Transduction efficiency after pre-treatment with eIF4A3-IN-1 for 1 hour, followed by infection with ssAAV2-luciferase for 24 hours.

FIG. 20C: Transduction efficiency after pre-treatment with eIF4A3-IN-1 for 2 hours, followed by infection with ssAAV2-luciferase for 24 hours.

FIG. 20D: Transduction efficiency after pre-treatment with eIF4A3-IN-1 for 4 hours, followed by infection with ssAAV2-luciferase for 24 hours.

FIG. 20E: Transduction efficiency after pre-treatment with eIF4A3-IN-1 for 6 hours, followed by infection with ssAAV2-luciferase for 24 hours.

FIG. 21A: Scheme of experiment investigating eIF4A inhibition on AAV transduction in other AAV serotypes. Multiplicity of Infection (MOI) is 20,000.

FIG. 21B: Transduction efficiency after pre-treatment with eIF4A3-IN-1 for 6 hours, followed by infection with ssAAV1-luciferase for 24 hours.

FIG. 21C: Transduction efficiency after pre-treatment with eIF4A3-IN-1 for 6 hours, followed by infection with ssAAV3b-luciferase for 24 hours.

FIG. 21D: Transduction efficiency after pre-treatment with eIF4A3-IN-1 for 6 hours, followed by infection with ssAAV5-luciferase for 24 hours.

FIG. 21E: Transduction efficiency after pre-treatment with eIF4A3-IN-1 for 6 hours, followed by infection with ssAAV9-luciferase for 24 hours.

FIG. 22A: Scheme of experiment investigating eIF4A inhibition on ssAAV-gaussia luciferase and scAAV-gaussia luciferase transduction. Multiplicity of Infection (MOI) is 10,000.

FIG. 22B: Transduction efficiency after pre-treatment with eIF4A3-IN-1 for 6 hours, followed by infection with ssAAV2-gaussia luciferace or scAAV2-gaussia luciferase for 24 hours.

FIG. 22C: Western blot analysis of eIF4A3 levels with respect to GAPDH levels in Hela cells pretreated with eIF4A3-IN-1 and infection with either ssAAV2-gaussia luciferace or scAAV2-gaussia luciferace.

FIG. 23A: Scheme of experiment investigating cell viability under zotatifin treatment.

FIG. 23B: Cell viability with 6 hours of zotatifin treatment.

FIG. 24A: Scheme of experiment investigating AAV transduction under zotatifin treatment.

FIG. 24B: Transduction efficiency after pre-treatment with Zotatifin for 6 hours, followed by infection with ssAAV2-luciferase for 24 hours.

FIG. 24C: Transduction efficiency after pre-treatment with Zotatifin for 6 hours, followed by infection with ssAAV1-luciferase for 24 hours.

FIG. 24D: Transduction efficiency after pre-treatment with Zotatifin for 6 hours, followed by infection with ssAAV3b-luciferase for 24 hours.

FIG. 24E: Transduction efficiency after pre-treatment with Zotatifin for 6 hours, followed by infection with ssAAV5-luciferase for 24 hours.

FIG. 24F: Transduction efficiency after pre-treatment with Zotatifin for 6 hours, followed by infection with ssAAV9-luciferase for 24 hours.

FIG. 25A: Scheme of experiment investigating eIF4A1, 2, and 3 activity inhibition on ssAAV2-gaussia luciferace and scAAV2-gaussia luciferace transduction. Multiplicity of Infection (MOI) is 20,000.

FIG. 25B: Transduction efficiency after pre-treatment with Zotatifin for 6 hours, followed by infection with ssAAV2-gaussia luciferace for 24 hours.

FIG. 25C: Western blot analysis of eIF4A3 levels with respect to GAPDH levels in HeLa cells pretreated with Zotatifin and infection with either ssAAV2-gaussia luciferace or scAAV2-gaussia luciferace.

### DETAILED DESCRIPTION

A description of example embodiments follows.

The present disclosure relates to host protein inhibitors or stereoisomers, tautomers or salts thereof. The host protein inhibitors may increase vector (e.g., a viral vector) transduction. Various steps are involved in the infectious pathway and transduction of vectors (e.g., adeno-associated virus (AAV) vectors) including but not limited to vector binding/attachment, uptake/internalization, cellular trafficking, nuclear entry, vector uncoating, DNA synthesis, and transgene transcription (see Dhungel, B. P. et al., Trends in Molecular Medicine, 2021, 27(2), pp. 172-184; Pillay, S. et al., Nature, 2016, 530(7588), pp. 108-112; Madigan, V. J. et al., PLOS pathogens, 2019, 15 (8), p.e1007988; and U.S. patent application Ser. No. 18/345,183, the foregoing are incorporated herein by reference in their entireties). As used herein, host proteins are any proteins that are expressed endogenously in a host organism and are involved in the transduction of a vector (e.g., a viral vector). In some embodiments, the host protein is host cellular machinery. Non-limiting examples of host proteins include proteins involved in endocytosis (e.g., RAB5, RAB7, and RAB10), nuclear import (e.g., NUP98, NUP153, Importin-5, and KPNB1), genome replication and transcription (HSP90, POLII, P14 and SNRP70), nuclear export (NXF1, Hsc79, and CRM1), translation (GRSF-1, P58IPK), posttranslational modification, protein transport/cell motor proteins (e.g., actin, and RAB11) (see U.S. patent application Ser. No. 17/155,764, which is incorporated herein by reference in its entirety). In some embodiments, the host protein is a cell surface moiety. A cell surface moiety may be any moiety present on the surface of a cell with which a vector interacts either directly or indirectly. A vector may interact directly with the cell surface moiety (e.g., a receptor on the surface of the cell) as a means to invade the cell. Non-limiting examples of cell surface moieties include common viral receptors such as sialylated glycans; cell adhesion molecules such as immunoglobulin superfamily members and integrins; and phosphatidylserine receptors.

In some embodiments, the host protein is a cell motor protein. In some embodiments the host protein is a protein transport protein (e.g., a cytoplasmic dynein cell motor protein, a kinesin protein, a microtubule protein such as TUBA1A or TUBB3).

Non-limiting examples of host proteins associated with the endosome include RAB5A, RAB4, EEA1, RAB7, RAB9, RAB 11, CLTB, and CTLC (see Russell et al., Curr. Opin. Cell Bio., 18(4): 422-428 (2006), which is incorporated herein by reference in its entirety). Non-limiting examples of host proteins associated with the endocytic pathway are described in Grant and Donaldson, Nat. Rev. Mol. Cell Biol., 10(9): 597-608 (2009), which is incorporated by reference herein in its entirety.

Table 1 provides non-limiting examples of host proteins, their corresponding description and functions. In some embodiments, the host protein is a member selected from the host proteins of Table 1. In some embodiments, the host protein is eIF4A3.

TABLE 1
Host proteins

| Original | | NCBI | | |
|---|---|---|---|---|
| No. | Name | Gene ID | Description | Functions |
| 13 | EIF4A3 | 9775 | eukaryotic translation | Involved in pre-mRNA |
| | | | initiation factor 4A3 | splicing, translation initiation |
| 17 | RAP1A | 5906 | RAP1A, member of RAS | regulates signaling pathways |
| | | | oncogene family | that affect cell proliferation and |
| | | | | adhesion |
| 18 | NIFK | 84365 | nucleolar protein interacting | may bind RNA and may play a |
| | | | with the FHA domain of | role in mitosis and cell cycle |
| | | | MKI67 | progression |
| 31 | SART1 | 9092 | spliceosome associated | Plays a role in mRNA splicing |
| | | | factor 1, recruiter of | as a component of the U4/U6- |
| | | | U4/U6.U5 tri-snRNP | U5 tri-snRNP |
| 10 | PLS1 | 5357 | plastin1 | Actin-bundling protein |
| 19 | SF3B6 | 51639 | splicing factor 3b subunit 6 | subunit of the splicing factor 3b |
| | | | | complex |

In some embodiments, the host protein inhibitor is

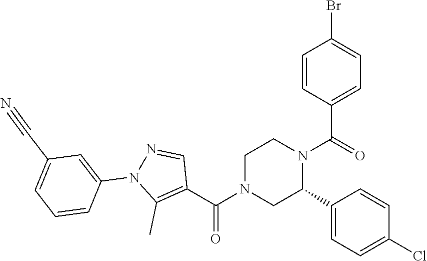 [SMILES: Cc1c(C(=O)N2CCN(C(=O)c3ccc(Br)cc3)[C@H](c3ccc(Cl)cc3)C2)cnn1-c1cccc(C#N)c1]

(3-[4-[(3R)-4-(4-bromobenzoyl)-3-(4-chlorophenyl)piperazine-1-carbonyl]-5-methylpyrazol-1-yl]benzonitrile, eIF4A3-IN-1),

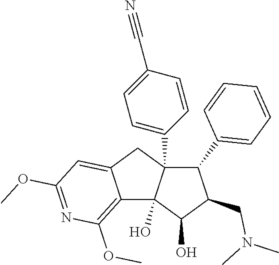

(4-[(2S,3R,4S,5S,6R)-4-[(dimethylamino)methyl]-2,3-dihydroxy-10,12-dimethoxy-5-phenyl-7-oxa-11-azatricyclo[6.4.0.02,6]dodeca-1(12),8,10-trien-6-yl]benzonitrile, Zotatifin), or a combination thereof.

In some embodiments, the host protein inhibitor is

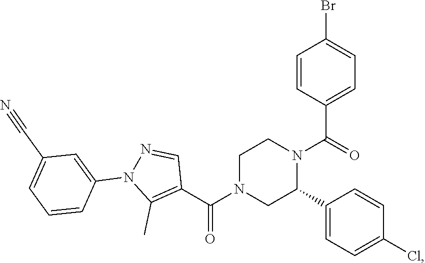 [SMILES: Cc1c(C(=O)N2CCN(C(=O)c3ccc(Br)cc3)[C@H](c3ccc(Cl)cc3)C2)cnn1-c1cccc(C#N)c1]

or a stereoisomer, tautomer or salt thereof.

In some embodiments, the host protein inhibitor is

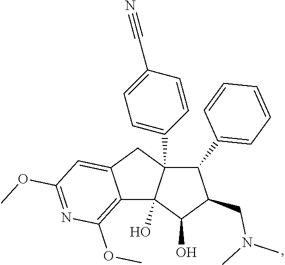

or a stereoisomer, tautomer or salt thereof.

The present disclosure also relates to vectors (e.g., viral vectors) comprising a transgene. The transgene (e.g., heterologous nucleic acid sequence) may be operably linked to regulatory sequences which direct expression of a product from the transgene in a host (e.g., a host cell).

In some embodiments, the vector is a viral vector (e.g., a recombinant viral vector). In one embodiment, the viral vector is an adeno-associated virus (AAV) vector (e.g., a recombinant AAV). Non-limiting examples of viral vectors include DNA or RNA viral vectors including but not limited to retroviral vectors, herpes virus vectors, adenovirus vectors, lentivirus vectors, rabies virus vectors, lentiviral vectors, VSV vectors, vaccinia virus vectors, reovirus vectors, semliki forest virus, and sindbis virus vectors. In some embodiments, the vector is an AAV1, AAV2, AAV3b, AAV5, or AAV9 vector. In some embodiments, the viral vector is a self-complementary vector, a single-stranded vector, or a combination thereof. In some embodiments, the viral vector is a single-stranded vector. As used heroin, the term “self-complementary vector” refers to a vector containing a double-stranded vector genome generated by the absence of a terminal resolution site (TR) from one of the inverted terminal repeats (ITRs) of the vector. The absence of a TR prevents the initiation of replication at the vector terminus where the TR is not present.

In some embodiments, a recombinant AAV is produced by providing a packaging cell line with a viral vector, helper functions for generating a productive AAV infection, and AAV cap genes, where the viral vector comprises a 5′ AAV inverted terminal repeat (ITR), a 3′ AAV ITR and a transgene; and recovering a recombinant AAV virus from the supernatant of the packaging cell line.

In some embodiments, the helper functions are provided by one or more helper plasmids or helper viruses comprising adenoviral helper genes. Non-limiting examples of the adenoviral helper genes include E1A, E1B, E2A, B4 and VA, which can provide helper functions to AAV packaging. Helper viruses of AAV are known in the art and include, for example, viruses from the family Adenoviridae and the family Herpesviridae.

In some embodiments, the vector is a non-viral vector. Non-viral vectors can be plasmid DNA, liposome-DNA complexes (lipoplexes), and polymer-DNA complexes (polyplexes). Non-viral vectors can be plasmid RNA, liposome-RNA complexes (lipoplexes), and polymer-RNA complexes (polyplexes). Oligonucleotides and their analogues, either alone or in complexes, are also possible non-viral vector-mediated gene transfer constructs.

As used herein, the transgene is a nucleic acid sequence, heterologous to the vector sequences flanking the transgene, which encodes a polypeptide, protein, or other product (e.g., RNA), of interest. The nucleic acid coding sequence is operatively linked to regulatory components in a manner which permits transgene transcription, translation, and/or expression in a host cell.

The size of the transgene can vary. For example, the transgene can be at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, or at least about 3.5 kb in length.

The transgene sequence may depend on the use of the resulting vector. For example, one type of transgene sequence includes a reporter sequence, which upon expression produces a detectable signal. Such reporter sequences include, without limitation, DNA sequences encoding β-lactamase, β-galactosidase (LacZ), alkaline phosphatase, thymidine kinase, green fluorescent protein (GFP), enhanced GFP (EGFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc. These coding sequences, when associated with regulatory elements which drive their expression, provide signals detectable by conventional means, including enzymatic, radiographic, colorimetric, fluorescence of other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

In some embodiments, the transgene is a non-marker sequence encoding a product (e.g., an expression product), such as but not limited to proteins, peptides, RNA, enzymes, dominant negative mutants, or catalytic RNAs. For example, proteins include an antibody or fragment thereof (e.g., an antigen-binding fragment of an antibody), and RNA molecules include, without limitation, messenger RNA (mRNA), tRNA, dsRNA, ribosomal RNA, small activating RNA (saRNA), catalytic RNAs, small interfering RNA (siRNA), small hairpin RNA, trans-splicing RNA, microRNA (miRNA), and antisense RNAs. The product may trigger gene inhibition or increase target gene expression. Another example of an RNA is a sequence which inhibits or abrogates expression of a targeted nucleic acid sequence (e.g., oncologic targets and viral disease targets) in a subject. These targeted nucleic acid sequences may be oncologic targets and viral disease targets. In some embodiments, the product is siRNA (e.g., antiviral siRNA), microRNA (miRNA), or antisense oligonucleotides (ASOs) (e.g., inhibitory ASOs, splicing-modulatory ASOs). In some embodiments, the product is miRNA.

In some embodiments, the transgene may be used to correct or ameliorate gene deficiencies, which may include deficiencies in which normal genes are expressed at less than normal levels or deficiencies in which the functional gene product is not expressed. The transgene may provide a product to a cell which is not natively expressed in the cell type or in the host. The transgene sequence may encode a therapeutic protein or polypeptide which is expressed in a host cell. The present disclosure further includes using multiple transgenes. In some embodiments, a different transgene may be used to encode each subunit of a protein, or to encode different peptides or proteins. For example, multiple transgenes may be used when the size of the DNA encoding the protein subunit is large, e.g., for an immunoglobulin, the platelet-derived growth factor, or a dystrophin protein. In order for the cell to produce the multi-subunit protein, a cell may be infected with a recombinant virus containing each of the different subunits. In some embodiments, when the transgene is large, consists of multi-subunits, or when two transgenes are co-delivered, the AAV carrying the desired transgene(s) or subunits are co-administered to allow them to concatamerize in vivo to form a single vector genome. In some embodiments, a first AAV may carry an expression cassette which expresses a single transgene and a second AAV may carry an expression cassette which expresses a different transgene for co-expression in the host cell.

Other non-limiting examples of transgene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other non-limiting examples of transgene sequences include antisense molecules and catalytic nucleic acids, such as ribozymes, which could be used to reduce overexpression of a target.

Vectors of the present disclosure may further comprise regulatory sequences operably linked to transgene(s) and the regulatory sequences direct expression of a product from the transgene in a cell. Regulatory sequences (e.g., expression control elements) are operably linked to the transgene in a manner which permits its transcription, translation and/or expression in a cell transfected with the vector. As used herein, “operably linked” sequences include both regulatory sequences that are contiguous with the transgene and regulatory sequences that act in trans or at a distance to control the transgene.

Regulatory sequences include but are not limited to appropriate transcription initiation, termination, promoter and enhancer sequences; efficient RNA processing signals such as splicing and polyadenylation (poly A) signals; sequences that stabilize cytoplasmic mRNA; sequences that enhance translation efficiency (i.e., Kozak consensus sequence); sequences that enhance protein stability; and when desired, sequences that enhance secretion of the encoded product. Regulatory sequences, including but not limited to promoters which are native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985), which is incorporated herein by reference in its entirety), the SV40 promoter, the dihydrofolate reductase promoter, the β-actin promoter, the phosphoglycerol kinase (PGK) promoter, and the EF1 promoter [Invitrogen]. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad.

Regulatory sequences include non-promoter regulatory elements. As used herein, a “non-promoter regulatory element” refers to non-promoter sequence(s) of a nucleic acid molecule that are capable of increasing or decreasing the expression of specific genes within the vector. Such non-promoter regulatory elements include but are not limited to, e.g., enhancer elements, inducer elements, silencer elements, 5′ untranslated regions (UTRs), 3′UTRs, terminator elements, CAAT boxes, CCAAT boxes, Pribnow boxes, SECIS elements, polyadenylation signals, A-boxes, Z-boxes, C-boxes, E-boxes, G-boxes, and Cis-regulatory elements (CREs). In some embodiments, the non-promoter regulatory element comprises an enhancer element. In some embodiments, the non-promoter regulatory element comprises an inducer element.

In some embodiments, the vector comprises the native promoter for the transgene. For example, the native promoter may be used when expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression. In another non-limiting example, if expression in skeletal muscle is desired, a promoter active in muscle should be used.

Vectors of the present disclosure may also comprise a selectable marker element or a reporter gene such as, without limitation, sequences encoding geneticin, hygromicin or purimycin resistance, among others. A “selectable marker element” is an element that confers a trait suitable for artificial selection. Such selectable marker elements or reporter genes may be used to signal, for example, the presence of plasmids in bacterial cells, such as ampicillin resistance. Vectors may further include an origin of replication. Examples of selectable marker elements useful in the present invention include, but are not limited to, beta-lactamase, neomycin resistance genes, mutant Fabl genes conferring triclosan resistance, URA3 elements, fluorescent gene products, affinity tags such as GST, His, CBP, MBP, and epitope tags such as Myc HA, FLAG. Selectable marker elements can be negative or positive selection markers. Vectors of the present disclosure may also include a cloning site. As used herein, a “cloning site” refers to a short segment of nucleotides in the vector that contain one or more unique restriction sites that allow for insertion of a nucleotide “target gene” or “gene of interest” into the vector.

Reduction and/or modulation of expression of a gene may be particularly desirable for treatment of hyperproliferative conditions characterized by hyperproliferating cells, such as but not limited to cancers and psoriasis. For example, target polypeptides include polypeptides which are produced exclusively or at higher levels in hyperproliferative cells as compared to normal cells, and target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include but are not limited to variable regions of antibodies made by B cell lymphomas and variable regions of T cell receptors of T cell lymphomas which, in some embodiments, are also used as target antigens for autoimmune disease.

Other non-limiting examples of products encoded by a transgene include hormones and growth and differentiation factors, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase. Yet other non-limiting examples of transegene products include proteins that regulate the immune system including, without limitation, cytokines and lymphokines such as thrombopoietin (TPO), interleukins (IL) IL-1 through IL-25 (including, e.g., IL-2, IL-4, IL-12 and IL-18), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Still other non-limiting examples of transgene products include any one of the receptors for the hormones, growth factors, cytokines, lymphokines, regulatory proteins and immune system proteins. The present disclosure encompasses receptors for cholesterol regulation and/or lipid modulation, including the low density lipoprotein (LDL) receptor, high density lipoprotein (HDL) receptor, the very low density lipoprotein (VLDL) receptor, and scavenger receptors. The present disclosure also encompasses gene products such as members of the steroid hormone receptor superfamily including glucocorticoid receptors and estrogen receptors, Vitamin D receptors and other nuclear receptors. In addition, other transgene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other transgene products include but are not limited to, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other transgene products include enzymes that may be used in enzyme replacement therapy for a variety of conditions resulting from deficient activity of enzyme. For example, enzymes that contain mannose-6-phosphate may be utilized in therapies for lysosomal storage diseases (e.g., a suitable gene includes that encoding 0-glucuronidase (GUSB))

### Pharmaceutical Compositions, Combinations, and Administration

Provided herein are pharmaceutical compositions comprising a vector (e.g., a viral vector), a host protein inhibitor or a stereoisomer, tautomer or salt thereof, and a pharmaceutically acceptable carrier or excipient. In some embodiments, the host protein is eIF4A1, eIF4A2, eIF4A3, RAP1A, NIFK, SART1, PLS1, or SF3B6. In some embodiments, the vector comprises a transgene (e.g., heterologous nucleic acid sequence) operably linked to regulatory sequences which direct expression of a product from the transgene in a cell. In certain embodiments, a pharmaceutical composition provided herein comprises a therapeutically and/or prophylactically effective amount of a host protein inhibitor or a stereoisomer, tautomer or salt thereof. In certain embodiments, the pharmaceutical composition comprises a therapeutically effective amount of a host protein inhibitor or a stereoisomer, tautomer or salt thereof. The pharmaceutical compositions provided herein may further comprise one or more additional therapeutic agents (e.g., anti-proliferative agents, e.g., anti-cancer agents).

Pharmaceutical compositions described herein can be prepared by any suitable method known in the art of pharmacology. In general, such preparatory methods include bringing a compound described herein (i.e., the “active ingredient”) into association with a carrier or excipient, and/or one or more other accessory ingredients, and then, if necessary and/or desirable, shaping, and/or packaging the product into a desired single- or multi-dose unit. In some embodiments, pharmaceutical compositions are adapted for oral administration.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient (e.g., host protein inhibitor or a stereoisomer, tautomer or salt thereof), the pharmaceutically acceptable carrier or excipient, and/or any additional ingredients in a pharmaceutical composition described herein will vary, depending, for example, upon the identity, size, and/or condition of the subject treated and upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Examples of diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Examples of granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Examples of surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Examples of binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Examples of preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Examples of antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Examples of chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Examples of antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Examples of antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Examples of alcohol preservatives include ethanol, polyethylene glycol, phenol, phenolic compounds, bisphenol, chlorobutanol, hydroxybenzoate, and phenylethyl alcohol.

Examples of acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Examples of buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Examples of lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Examples of natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms, for example, for oral and parenteral administration, include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the active ingredient is mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents, such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface-active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein.

A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

A therapeutic agent (e.g., a host protein inhibitor or a stereoisomer, tautomer or salt thereof, a vector) described herein, or a composition thereof, can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), ophthalmic, mucosal, nasal, bucal, sublingual; by intratracheal instillation, bronchial instillation, and/or inhalation; and/or as an oral spray, nasal spray, and/or aerosol. Specifically, contemplated routes are oral administration, intravenous administration (e.g., systemic intravenous injection), regional administration via blood and/or lymph supply, and/or direct administration to an affected site. In general, the most appropriate route of administration will depend upon a variety of factors, such as the nature of the agent (e.g., its stability in the environment of the gastrointestinal tract), and/or the condition of the subject (e.g., whether the subject is able to tolerate oral administration). In some embodiments, a pharmaceutical composition is formulated for oral administration.

In certain instances, it may be advantageous to administer a compound or agent of the present disclosure (e.g., a host protein inhibitor or a stereoisomer, tautomer or salt thereof, a vector) in combination with one or more additional therapeutic agent(s). For example, it may be advantageous to administer a compound of the present disclosure in combination with one or more additional therapeutic agents, e.g., independently selected from an anti-cancer agent (e.g., chemotherapeutic agent), immunotherapy (e.g., an immune checkpoint inhibitor), anti-allergic agent, anti-emetic, pain reliever, immunomodulator and cytoprotective agent, to treat cancer.

Compositions for use in combination therapies will either be formulated together as a pharmaceutical combination, or provided for separate administration (e.g., associated in a kit). Accordingly, provided herein is a pharmaceutical combination comprising a compound of the present disclosure (e.g., a host protein inhibitor or a stereoisomer, tautomer or salt thereof) (e.g., a therapeutically effective amount of a compound of the present disclosure), and one or more other therapeutic agents (e.g., a therapeutically effective amount of one or more other therapeutic agents). A pharmaceutical combination can further comprise one or more pharmaceutically acceptable carriers or excipients, such as one or more of the pharmaceutically acceptable carriers or excipients described herein. Additional therapeutic agents for the pharmaceutical combinations and kits described herein include any of the therapeutic agents identified herein, particularly with respect to combination therapies, discussed below.

Therapeutic agents, such as the compounds and compositions described herein, are typically formulated in dosage unit form for ease of administration and uniformity of dosage. It will be understood, however, that the total daily usage of such forms will be decided by a physician within the scope of sound medical judgment. The specific therapeutically effective dose level for any particular subject or organism will depend upon a variety of factors including, for example, the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a therapeutic agent in a composition required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses may include different or substantially the same amounts of a therapeutic agent, such as a compound described herein. In certain embodiments, when multiple doses are administered to a subject or applied to a tissue or cell, the frequency of administering the multiple doses to the subject or applying the multiple doses to the tissue or cell is three doses per day, two doses per day (e.g. BID), one dose per day (e.g., QD), one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In certain embodiments, a dose (e.g., a single dose, or any dose of multiple doses, a unit dosage form) includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose includes independently between 1 mg and 3 mg, inclusive, of a compound described herein. In certain embodiments, a dose includes independently between 3 mg and 10 mg, inclusive, of a compound described herein. In certain embodiments, a dose includes independently between 10 mg and 30 mg, inclusive, of a compound described herein. In certain embodiments, a dose includes independently between 30 mg and 100 mg, inclusive, of a compound described herein. In certain embodiments, a dose includes independently between 10 mg and 250 mg, inclusive, of a compound described herein. In certain embodiments, a dose includes independently between 10 mg and 100 mg (e.g., about 45 mg, about 75 mg, about 90 mg), inclusive, of a compound described herein.

For example, the pharmaceutical compositions or combinations described herein can be in a unit dosage form containing from about 1 to about 1000 mg of active ingredient(s) (e.g., for a subject of from about 50 to about 70 kg), or from about 1 to about 500 mg, from about 1 to about 250 mg, from about 1 to about 150 mg, from about 0.5 to about 100 mg, or from about 1 to about 50 mg of active ingredient(s) (e.g., for a subject of from about 50 to about 70 kg). The therapeutically effective dosage of a compound, pharmaceutical composition or pharmaceutical combination is dependent on the species of the subject, the body weight, age and individual condition of the subject, and the disease, disorder or condition or the severity thereof being treated. A physician, clinician or veterinarian of ordinary skill can readily determine the therapeutically effective amount of each of the active ingredients necessary to prevent or treat the progress of the disease, disorder or condition.

Compositions can also be formulated so as to deliver a particular dose to a subject. A dose may range, depending on the route of administration, among other things, between about 0.1 mg/kg to about 500 mg/kg subject mass, or between about 1 mg/kg to about 100 mg/kg subject mass. In some embodiments, the dosage is expected to be in the range of 1 mg/Kg subject mass and 150 mg/Kg subject mass, for example, at least about 1 mg/Kg, at least about 10 mg/Kg, at least about 20 mg/Kg, at least about 30 mg/Kg, at least about 40 mg/Kg, at least about 50 mg/Kg, at least about 60 mg/Kg, at least about 70 mg/Kg, at least about 80 mg/Kg, at least about 90 mg/Kg, at least about 100 mg/Kg, at least about 110 mg/Kg, at least about 120 mg/Kg, at least about 130 mg/Kg, at least about 140 mg/Kg, or about 150 mg/Kg.

In some embodiments, dose ranges described herein provide guidance for the administration of provided pharmaceutical compositions to an adult. The amount to be administered to, for example, a child or an adolescent, can be determined by a medical practitioner or person skilled in the art and can be lower or the same as that administered to an adult.

### Kits

Also encompassed by the disclosure are kits (e.g., pharmaceutical packs). The kits provided may comprise a compound (e.g., an inhibitor) of the disclosure, or pharmaceutical composition thereof, and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound contained in the kit. In some embodiments, the pharmaceutical composition or compound described herein provided in the first container and the second container are combined to form a unit dosage form.

Thus, in one aspect, provided are kits including a first container comprising host protein inhibitors of the disclosure, or a pharmaceutical composition thereof. In certain embodiments, the kits are useful in one or more of the methods described herein, for example, for treating a disease (e.g., a proliferative disease such as cancer) in a subject in need thereof. In certain embodiments, the kits are useful for preventing a disease in a subject in need thereof. In certain embodiments, the kits are useful for reducing the risk of developing a disease in a subject in need thereof.

A kit described herein may include one or more additional therapeutic agents (e.g., viral vector) described herein as a separate composition or in a combination comprising a compound (e.g., host protein inhibitors) of the disclosure, or pharmaceutical composition thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information.

In the combinations and/or kits described herein, the compound of the present disclosure and the other therapeutic agent may be manufactured and/or formulated by the same or different manufacturers. Moreover, the compound of the present disclosure and the other therapeutic agent may be brought together into a combination therapy: (i) prior to release of the combination product to physicians (e.g., in the case of a kit comprising the compound of the present disclosure and the other therapeutic agent); (ii) by the physician (or under the guidance of a physician) shortly before administration; (iii) in the patient themselves, e.g., during sequential administration of the compound of the present disclosure and the other therapeutic agent.

A pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

In some embodiments, the concentration of one or more therapeutic agents provided in a pharmaceutical composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more therapeutic agents provided in a pharmaceutical composition is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more therapeutic agents provided in a pharmaceutical composition is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v.

In some embodiments, the concentration of one or more therapeutic agents provided in a pharmaceutical composition is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

### Methods and Uses

In some embodiments, the present disclosure provides methods of increasing transduction efficiency of a vector (e.g., a viral vector) to a cell (e.g., a host cell), comprising contacting the cell with a a host protein inhibitor or a stereoisomer, tautomer or salt thereof. In some embodiments, transduction efficiency of a vector to a cell is measured based on luciferase activity from the vector expressing the luciferase reporter gene. In some embodiments, the host protein is eIF4A1, eIF4A2, eIF4A3, RAP1A, NIFK, SART1, PLS1, or SF3B6. In some embodiments, the vector comprises a transgene (e.g., heterologous nucleic acid sequence) operably linked to regulatory sequences which direct expression of a product from the transgene in the cell.

In some embodiments, the cell is a mammalian cell. In some embodiments, the cell comprises an in vitro cell, an ex vivo cell, or both. In some embodiments, the cell is a neuronal cell. In some embodiments, the cell is a cancer cell. In some embodiments, the cell is a cancer cell or neuronal cell of a subject.

In some embodiments, the present disclosure provides methods of delivering a heterologous nucleic acid sequence to a cell in a subject, said method comprising administering to the subject a pharmaceutical composition comprising a vector and a host protein inhibitor or a stereoisomer, tautomer or salt thereof. In some embodiments, the host protein is eIF4A1, eIF4A2, eIF4A3, RAP1A, NIFK, SART1, PLS1, or SF3B6. In some embodiments, the vector comprises a transgene (e.g., heterologous nucleic acid sequence) operably linked to regulatory sequences which direct expression of a product from the transgene in the cell. In some embodiments, the cell is a cancer cell.

In some embodiments, methods of the present disclosure may further comprise assaying a sample from a subject or a host for the presence of antibodies to a selected AAV source (e.g., a serotype). A variety of assay formats for detecting neutralizing antibodies are well known to those of skill in the art. The results of the assay may be used to determine which AAV vector containing capsid proteins of a particular source are preferred for delivery, e.g., by the absence of neutralizing antibodies specific for that capsid source.

In some embodiments, multiple vectors may be used to deliver large transgenes or multiple transgenes by co-administration of vectors concatamerize in vivo to form a single vector genome. In some embodiments, a first vector may carry an expression cassette which expresses a single transgene (or a subunit thereof) and a second vector may carry an expression cassette which expresses a second transgene (or a different subunit) for co-expression in the host cell. A first vector may carry an expression cassette which is a first piece of a polycistronic construct (e.g., a promoter and transgene, or subunit) and a second vector may carry an expression cassette which is a second piece of a polycistronic construct (e.g., transgene or subunit and a polyA sequence). The two pieces of a polycistronic construct may concatamerize in vivo to form a single vector genome that co-expresses the transgenes delivered by the first and second vector. In some embodiments, the vector carrying the first expression cassette and the vector carrying the second expression cassette may be delivered in a single pharmaceutical composition. In some embodiments, the two or more vectors are delivered as separate pharmaceutical compositions which can be administered substantially simultaneously, or shortly before or after one another.

In some embodiments, vectors (e.g., viral vectors) and compounds provided herein (e.g., host protein inhibitors) are useful for treating and/or preventing diseases (e.g., fibrotic diseases, for example IPF or cardiac fibrosis or a cardiac disease associated with TGFβ signaling, and proliferative diseases, e.g., a cancer) in a subject (e.g., a subject in need thereof), inhibiting tumor growth in a subject (e.g., a subject in need thereof), or inhibiting the activity of a host protein in vitro or in vivo. In some embodiments, the compounds of the disclosure are useful in moderating, preventing, or providing treatment for conditions and/or diseases the progress of which is driven by, or utilizes the host proteins for disease progression.

Provided herein are methods of treating and/or preventing (e.g., treating) a disease, disorder or condition described herein (e.g., a fibrotic disease which is present by itself or comorbid with an infectious, inflammatory or proliferative disease (either benign or malignant), or a proliferative disease, e.g., cancer) in a subject (e.g., a subject in need thereof), the methods comprising administering to the subject a therapeutically and/or prophylactically effective amount (e.g., therapeutically effective amount) of a compound (e.g., a host protein inhibitor or a stereoisomer, tautomer or salt thereof), or a pharmaceutical composition thereof (e.g., a pharmaceutical composition comprising a vector (e.g., a viral vector) and a host protein inhibitor or a stereoisomer, tautomer or salt thereof). Also provided herein are compounds and compositions for use in treating and/or preventing a disease, disorder or condition described herein (e.g., a fibrotic disease which is present by itself or comorbid with an infectious, inflammatory or proliferative disease (either benign or malignant), or a proliferative disease, e.g., cancer). Also provided herein are uses of compounds (e.g., a host protein inhibitor or a stereoisomer, tautomer or salt thereof) and pharmaceutical compositions for the manufacture of a medicament for treating and/or preventing a disease, disorder or condition described herein (e.g., a fibrotic disease which is present by itself or comorbid with an infectious, inflammatory or proliferative disease (either benign or malignant), or a proliferative disease, e.g., cancer). In certain embodiments, the disease, disorder or condition is a disease, disorder or condition associated with host protein activity, e.g., in a subject or cell.

In some embodiments, the disease, disorder or condition is selected from the group consisting of cancer, ischemia, diabetic retinopathy, macular degeneration, rheumatoid arthritis, psoriasis, HIV infection, sickle cell anemia, Alzheimer's disease, muscular dystrophy, neurodegenerative disease, vascular disease, cystic fibrosis, and stroke. In some embodiments, the disease, disorder or condition is a neurodegenerative disease. In some embodiments, the disease, disorder or condition is a cancer.

In some embodiments, vectors (e.g., viral vectors) are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., the liver (optionally via the hepatic artery) or lung), oral, inhalation, intranasal, intratracheal, intraarterial, intraocular, intravenous, intramuscular, subcutaneous, intradermal, and other parental routes of administration. Routes of administration may be combined, if desired.

In some embodiments, dosages of the vector (e.g., a viral vector) depends on factors such as the condition being treated, the age, weight and health of the patient, and may thus vary among patients. For example, a therapeutically effective human dosage of the viral vector is in the range of from about 0.1 mL to about 100 mL of solution containing concentrations of from about 1×10⁹ to 1×10¹⁶ genomes virus vector. A non-limiting example of a human dosage for delivery to large organs (e.g., liver, muscle, heart and lung) may be about 5×10¹⁰ to 5×10¹³ AAV genomes per 1 kg, at a volume of about 1 to 100 mL. A non-limiting example of dosage for delivery to eye is about 5×10⁹ to 5×10¹² genome copies, at a volume of about 0.1 mL to 1 mL. The dosage may be adjusted to balance the therapeutic benefit against any side effects and such dosages may vary depending upon the therapeutic application for which the recombinant vector is employed. The levels of expression of the transgene may be monitored to determine the frequency of dosage resulting in vectors. Dosage regimens similar to those described for therapeutic purposes may also be utilized for immunization using compositions of the disclosure.

Vectors (e.g., viral vectors) may be used for therapeutic and/or vaccinal regimens. Additionally, vectors may be delivered in combination with one or more other vectors or active ingredients in a desired therapeutic and/or vaccinal regimen. Compositions of the present disclosure are well-suited to gene delivery for therapeutic purposes and for immunization, including inducing protective immunity. Vectors and compositions of the present disclosure may also be used in immunization regimens such as those described in U.S. Patent Application No. 60/565,936, filed Apr. 28, 2004 for “Sequential Adenovirus and AAV-Mediated Delivery of Immunogenic Molecules”, which is incorporated herein by reference in its entirety.

A “proliferative disease” refers to a disease that occurs due to abnormal growth or extension by the multiplication of cells (Walker, Cambridge Dictionary of Biology, Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases. In some embodiments, the proliferative disease is a hematological cancer (e.g., anaplastic large cell lymphoma (ALCL), myelodysplastic syndrome, multiple myeloma, and myelofibrosis).

In certain embodiments, the disease, disorder or condition to be treated is cancer. Provided herein are methods for treating cancer in a subject (e.g., a subject in need thereof), the methods comprising administering to the subject a therapeutically effective amount of a host protein inhibitor, or a pharmaceutical composition thereof.

A wide variety of cancers, including solid tumors, leukemias, lymphomas, and myelomas are amenable to the methods disclosed herein. In some embodiments, the cancer is a solid tumor cancer. In some embodiments, the cancer comprises a solid tumor (e.g., a colorectal, breast, prostate, lung, pancreatic, renal or ovarian tumor). Accordingly, in some embodiments, the cancer is a solid tumor cancer. In some embodiments, the cancer is selected from one or more of a cancer of the pulmonary system, a brain cancer, a cancer of the gastrointestinal tract, a skin cancer, a genitourinary cancer, head and neck cancer, a sarcoma, a carcinoma, and a neuroendocrine cancer. In various embodiments, the solid tumor cancer is breast cancer, bladder cancer, endometrial cancer, esophageal cancer, liver cancer, pancreatic cancer, lung cancer, cervical cancer, colon cancer, colorectal cancer, gastric cancer, kidney cancer, ovarian cancer, prostate cancer, testicular cancer, uterine cancer, a viral-induced cancer, melanoma or sarcoma. In some embodiments, the cancer is cervical cancer. In some embodiments, the cancer is bladder cancer. In some embodiments, the cancer is lung cancer (e.g., non-small cell lung cancer). In other embodiments, the cancer is liver cancer. In some embodiments, the cancer is a sarcoma, bladder cancer or renal cancer. In some embodiments, the cancer is gastric cancer. In some embodiments, the cancer is breast cancer. In some embodiments, the cancer is ovarian cancer. In some embodiments, the cancer is pancreatic cancer. In some embodiments, the cancer is mesothelioma. In some embodiments, the cancer is prostate cancer (e.g., castration-resistant prostate cancer, castration-sensitive prostate cancer). In other embodiments, the cancer is bladder cancer, pancreatic cancer, colorectal cancer, glioblastoma, kidney cancer, non-small cell lung carcinoma, prostate cancer, sarcoma, skin cancer, thyroid cancer, testicular cancer or vulvar cancer. In some embodiments, the cancer is endometrial cancer, pancreatic cancer, testicular cancer, renal cancer, melanoma, colorectal cancer, thyroid cancer, bladder cancer, pancreatic cancer, vulvar cancer, sarcoma, prostate cancer, lung cancer or anal cancer. In some embodiments, the cancer is a sarcoma. In some embodiments, the cancer is a renal cell carcinoma. In particular embodiments, the cancer is ovarian granulosa cell tumor (e.g., adult granulosa cell tumor (AGCT), pediatric granulosa cell tumor).

In some embodiments, the cancer is a non-solid tumor cancer. In some embodiments, the cancer is a hematologic cancer. Hematologic cancers that can be treated according to the methods described herein include leukemias (e.g., acute leukemias, chronic leukemias), lymphomas (e.g., B-cell lymphoma, T-cell lymphoma) and multiple myeloma. In some embodiments, the hematologic cancer is selected from multiple myeloma, myelodysplastic syndrome (MDS), acute myeloid leukemia (AML), acute lymphoblastic leukemia (ALL), acute lymphocytic leukemia, lymphocytic lymphoma, mycosis fungoides, chronic lymphogenous leukemia, chronic lymphocytic leukemia (CLL), mantle cell lymphoma, diffuse large B-cell lymphoma, follicular lymphoma, Hodgkin's lymphoma, non-Hodgkin's lymphoma or myelofibrosis.

Examples of cancer treatable according to the methods described herein include, but are not limited to, adenocarcinoma of the breast, prostate, and colon; all forms of bronchogenic carcinoma of the lung; myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, lung cancer (e.g., large cell lung cancer, such as squamous cell carcinoma, non-small cell lung), oat cell, papillary, scirrhous, bronchiolar, bronchogenic, squamous cell, and transitional cell). Additional examples of cancer treatable according to the methods described herein include, but are not limited to, histiocytic disorders; leukemia; histiocytosis malignant; Hodgkin's disease; hypereosinophilia, immunoproliferative small; non-Hodgkin's lymphoma; plasmacytoma; reticuloendotheliosis; melanoma; chondroblastoma; chondroma; chondrosarcoma; dermatofibrosarcoma protuberans, fibrotic cancer (myelofibrosis, pancreatic cancer (e.g., pancreatic ductal adenocarcinoma), kidney cancer, liver cancer, lung cancer (e.g., large cell lung cancer, such as squamous cell carcinoma), breast cancer (e.g., inflammatory breast cancer), ovarian cancer (e.g., high grade serious ovarian carcinoma), endometrial cancer, uterine cancer, uterine sarcoma (e.g., uterine leiomyosarcoma), renal cell cancer, sarcoma (e.g., soft tissue sarcoma), malignant fibrous histiocytoma, fibrosarcoma (e.g., dermatofibrosarcoma protuberans) and hepatocellular carcinoma); fibroma; fibrosarcoma; giant cell tumors; histiocytoma; lipoma; liposarcoma; mesothelioma; myxoma; myxosarcoma; osteoma; osteosarcoma; pediatric malignancy, chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatocellular cancer, hepatoma; hidradenoma; islet cell tumor; Leydig cell tumor; papilloma; sertoli cell tumor; theca cell tumor; leiomyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. Yet more examples of cancer treatable according to the methods described herein include, but are not limited to, angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; neurofibromatosis; and cervical dysplasia.

Gene therapy has been used to restore gene function in specific target cells in neurologic and neurodegenerative disorders (Deverman, Ravina et al. 2018). Gene transfer by systemic vector delivery via peripheral vascular transduction can be difficult for efficient expression in a neuron-specific or pan-neuronal fashion in the CNS (Ingusci, Verlengia et al. 2019). AAV vectors are among the most efficient vehicles to achieve gene expression in the CNS (Hudry and Vandenberghe 2019, Wang, Tai et al. 2019). Moreover, engineered AAV capsids have shown improved CNS transduction and enhanced capacity to cross the BBB with higher efficiency than naturally-occurring serotypes (Chan, Jang et al. 2017, Bedbrook, Deverman et al. 2018, Hordeaux, Yuan et al. 2019, Qin Huang and Alejandro B. Balazs 2019).

Non-limiting examples of neurodegenerative diseases include dementia, multiple sclerosis, Parkinson syndrome, juvenile parkinsonism, striatonigral degeneration, progressive supranuclear palsy, pure akinesia, prion disease, corticobasal degeneration, chorea-acanthocytosis, benign hereditary chorea, paroxysmal choreoathetosis, essential tremor, essential myoclonus, Gilles de la Tourette syndrome, Rett syndrome, degenerative ballism, dystonia musculorum deformans, athetosis, spasmodic torticollis, Meige syndrome, cerebral palsy, Wilson's disease, Segawa's disease, Hallervorden-Spatz syndrome, neuroaxonal dystrophy, pallidal atrophy, spinocerebellar degeneration, cerebral cortical atrophy, Holmes-type cerebellar atrophy, olivopontocerebellar atrophy, hereditary olivopontocerebellar atrophy, Joseph disease, dentatorubropallidoluysian atrophy, Gerstmann-Straussler-Scheinker syndrome, Friedreich ataxia, Roussy-Levy syndrome, May-White syndrome, congenital cerebellar ataxia, periodic hereditary ataxia, ataxia telangiectasia, amyotrophic lateral sclerosis, progressive bulbar palsy, spinal progressive muscular atrophy, spinobulbar muscular atrophy, Werdnig-Hoffmann disease, Kugelberg-Welander disease, hereditary spastic paraplegia, syringomyelia, syringobulbia, Arnold-Chiari malformation, stiff man syndrome, Klippel-Feil syndrome, Fazio-Londe disease, low myelopathy, Dandy-Walker syndrome, spina bifida, Sjogren-Larsson syndrome, radiation myelopathy, age-related macular degeneration, and cerebral apoplexy due to cerebral hemorrhage and/or dysfunction or neurologic deficits associated therewith.

Additionally, provided herein are methods of inhibiting tumor growth in a subject (e.g., a subject in need thereof), the methods comprising administering to the subject a therapeutically effective amount of a compound of the present disclosure (e.g., a host protein inhibitor), or a pharmaceutical composition thereof.

Further, the present disclosure also relates to methods of producing a gene product in vitro. For in vitro production, a gene product (e.g., a protein) may be obtained from a culture following transfection of host cells with a vector containing the molecule encoding the product and culturing the cell culture under conditions which permit expression. The expressed product may then be purified and isolated, as desired. Suitable techniques for transfection, cell culturing, purification, and isolation are known to those of skill in the art.

It will be readily understood that the aspects and embodiments, as generally described herein, are exemplary. The following more detailed description of various aspects and embodiments are not intended to limit the scope of the present disclosure, but is merely representative of various aspects and embodiments. Moreover, the compositions and methods disclosed herein may be changed by those skilled in the art without departing from the scope of the present disclosure. Unless defined otherwise, all technical and scientific terms used herein have the same meaning as is commonly understood by one of skill in the art to which this disclosure belongs. All publications and patents referred to herein are incorporated by reference.

### Definitions

For purposes of the present disclosure, the following definitions will be used unless expressly stated otherwise:

The terms “a”, “an”, “the” and similar referents used in the context of describing the present disclosure are to be construed to cover both the singular and the plural, unless otherwise indicated herein or clearly contradicted by context. All methods described herein, can be performed in any suitable order unless otherwise indicated herein or otherwise clearly contradicted by context. The use of any and all examples, or exemplary language (e.g., “such as”) provided herein, is intended merely to better illuminate the present disclosure and does not pose a limitation on the scope of the disclosure otherwise claimed. No language in the present specification should be construed as indicating any unclaimed element is essential to the practice of the disclosure.

The term “about” in relation to a given numerical value, such as for temperature and period of time, is meant to include numerical values within 10% of the specified value.

Adeno-associated virus is a nonpathogenic parvovirus composed of a 4.7 kb single-stranded DNA genome within a non-enveloped, icosahedral capsid. “AAV” is an abbreviation for adeno-associated virus, and may be used to refer to the virus itself or derivatives thereof. The genome contains three open reading frames (ORF) flanked by inverted terminal repeats (ITR) that function as the viral origin of replication and packaging signal. The rep ORF encodes four nonstructural proteins that play roles in viral replication, transcriptional regulation, site-specific integration, and virion assembly. The cap ORF encodes three structural proteins (VP1-3) that assemble to form a 60-mer viral capsid. Finally, an ORF present as an alternate reading frame within the cap gene produces the assembly-activating protein (AAP), a viral protein that localizes AAV capsid proteins to the nucleolus and functions in the capsid assembly process.

There are several naturally occurring serotypes and over 100 variants of AAV, each of which differs in amino acid sequence, particularly within the hypervariable regions of the capsid proteins, and thus in their gene delivery properties. No AAV has been associated with any human disease, making recombinant AAV attractive for clinical applications.

The term “AAV” as used herein covers all subtypes and both naturally occurring and recombinant forms, except where required otherwise. The term “AAV” includes AAV type 1 (AAV-1 or AAV1), AAV type 2 (AAV-2 or AAV2), AAV type 3 (AAV-3 or AAV3), AAV type 4 (AAV-4 or AAV4), AAV type 5 (AAV-5 or AAV5), AAV type 6 (AAV-6 or AAV6), AAV type 7 (AAV-7 or AAV7), AAV type 8 (AAV-8 or AAV8), AAV type 9 (AAV-9 or AAV9), avian AAV, bovine AAV, canine AAV, equine AAV, primate AAV, non-primate AAV, and ovine AAV. “Primate AAV” refers to AAV that infect primates, “non-primate AAV” refers to AAV that infect non-primate mammals, “bovine AAV” refers to AAV that infect bovine mammals, etc.

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_002077.1 (AAV-1), AF063497.1 (AAV-1), NC_001401.2 (AAV-2), AF043303.1 (AAV-2), J01901.1 (AAV-2), U48704.1 (AAV-3), NC_001729.1 (AAV-3), NC_001829.1 (AAV-4), U89790.1 (AAV-4), NC_006152.1 (AAV-5), AF085716.1 (AAV-5), AF028704.1 (AAV-6), NC_006260.1 (AAV-7), AF513851.1 (AAV-7), AF513852.1 (AAV-8) NC_006261.1 (AAV-8), and AY530579.1 (AAV-9); the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al., (1986) J. Virol. 58:921; Gao et al. (2002) Proc. Nat. Acad. Sci. USA 99:11854; Moris et al. (2004) Virology 33:375-383; international patent publications WO 00/28061, WO 99/61601, WO 98/11244; and U.S. Pat. No. 6,156,303.

The sequences of naturally existing cap (capsid) proteins associated with AAV serotypes are known in the art and include: AAV1 (SEQ ID NO: 1), AAV2 (SEQ ID NO: 2), AAV3 (SEQ ID NO: 3), AAV4 (SEQ ID NO: 4), AAV5 (SEQ ID NO: 5), AAV6 (SEQ ID NO: 6), AAV7 (SEQ ID NO: 7), AAV8 (SEQ ID NO: 8), and AAV9 (SEQ ID NO: 9). The term “variant AAV capsid protein” is a an AAV capsid protein comprising an amino acid sequence that includes at least one substitution (including deletion, insertion, etc.) relative to one of the naturally existing AAV capsid protein sequences set forth in SEQ ID NOs: 1-9.

An “AAV virion” or “AAV viral particle” refers to a viral particle composed of at least one AAV capsid protein and an encapsidated AAV polynucleotide.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

If an AAV virion comprises a heterologous polynucleotide (i.e. a polynucleotide other than a wild-type AAV genome, e.g., a transgene to be delivered to a target cell, an RNAi agent or CRISPR agent to be delivered to a target cell, etc.), it is typically referred to as a “recombinant AAV (rAAV) virion” or an “rAAV viral particle.” In general, the heterologous polynucleotide is flanked by at least one, and generally by two, AAV inverted terminal repeat sequences (ITRs).

The term “rAAV vector” encompasses rAAV virions (i.e., rAAV viral particles) (e.g., an infectious rAAV virion), which by definition include an rAAV polynucleotide; and also encompasses polynucleotides encoding rAAV (e.g., a single stranded polynucleotide encoding rAAV (ss-rAAV); a double stranded polynucleotide encoding rAAV (ds-rAAV), e.g., plasmids encoding rAAV; and the like).

“Packaging” refers to a series of intracellular events that result in the assembly and encapsidation of an AAV particle.

AAV “rep” and “cap” genes refer to polynucleotide sequences encoding replication and encapsidation proteins of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans. For example, a plasmid or other expression vector comprising nucleotide sequences encoding one or more adenoviral proteins is transfected into a producer cell along with an rAAV vector.

An “infectious” virus or viral particle is one that comprises a competently assembled viral capsid and is capable of delivering a polynucleotide component into a cell for which the viral species is tropic. The term does not necessarily imply any replication capacity of the virus. Assays for counting infectious viral particles are described elsewhere in this disclosure and in the art. Viral infectivity can be expressed as the ratio of infectious viral particles to total viral particles. Methods of determining the ratio of infectious viral particle to total viral particle are known in the art. See, e.g., Grainger et al. (2005) Mol. Ther. 11: S337 (describing a TCID50 infectious titer assay); and Zolotukhin et al. (1999) Gene Ther. 6:973. See also the Examples.

The term “tropism” as used herein refers to the preferential targeting of specific host species or specific cell types within a host species by a virus (e.g., an AAV). For example, a virus that can infect cells of the heart, lung, liver, and muscle has a broader (i.e., increased) tropism relative to a virus that can infect only lung and muscle cells. Tropism can also include the dependence of a virus on particular types of cell surface molecules of the host. For example, some viruses can infect only cells with surface glycosaminoglycans, while other viruses can infect only cells with sialic acid (such dependencies can be tested using various cells lines deficient in particular classes of molecules as potential host cells for viral infection). In some cases, the tropism of a virus describes the virus's relative preferences. For example, a first virus may be able to infect all cell types but is much more successful in infecting those cells with surface glycosaminoglycans. A second virus can be considered to have a similar (or identical) tropism as the first virus if the second virus also prefers the same characteristics (e.g., the second virus is also more successful in infecting those cells with surface glycosaminoglycans), even if the absolute transduction efficiencies are not similar. For example, the second virus might be more efficient than the first virus at infecting every given cell type tested, but if the relative preferences are similar (or identical), the second virus can still be considered to have a similar (or identical) tropism as the first virus. In some embodiments, the tropism of a virion comprising a subject variant AAV capsid protein is not altered relative to a naturally occurring virion. In some embodiments, the tropism of a virion comprising a subject variant AAV capsid protein is expanded (i.e., broadened) relative to a naturally occurring virion. In some embodiments, the tropism of a virion comprising a subject variant AAV capsid protein is reduced relative to a naturally occurring virion.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). Additionally, encompassed are compounds as individual isomers substantially free of other isomers, and alternatively, as mixtures of various isomers.

Unless otherwise stated, structures depicted herein are also meant to include compounds that differ only in the enrichment of the compound with one or more isotopes, for example, compounds having the present structures except selected positions occupied by hydrogen are enriched with deuterium or tritium, selected positions occupied by F are enriched by ¹⁹F, or selected positions occupied by C are enriched by ¹³C or ¹⁴C. Examples of isotopes that can be incorporated into compounds described herein include isotopes of hydrogen, carbon, nitrogen, oxygen, phosphorous, fluorine, chlorine and iodine, such as ²H, ³H, ¹¹C, ¹³C, ¹⁴C, ¹⁵N, ¹⁸F, ³¹P, ³²P, ³⁵S, ³⁶Cl, ¹²³I, 124I and ¹²⁵I, respectively. It will be appreciated that numerous other isotopic enrichments may be made and be within the scope of the present invention and are within the scope of the disclosure. Such compounds are useful, for example, as therapeutics or as analytical tools or probes in biological assays.

As used herein, the term “isomers” refers to different compounds that have the same molecular formula but differ in arrangement and configuration of the atoms.

“Enantiomers” are a pair of stereoisomers that are non-superimposable mirror images of each other. A 1:1 mixture of a pair of enantiomers is a “racemic” mixture. “Racemate” or “racemic” is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g., (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Geometric isomers may occur when a compound contains a double bond or some other feature that gives the molecule a certain amount of structural rigidity. If the compound contains a double bond, the double bond may be E- or Z-configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single bond.

The term “atropisomer,” as used herein, refers to a structural isomer based on axial or planar chirality resulting from restricted rotation in the molecule.

Optically active (R)- and(S)-isomers may be prepared using chiral synthons or chiral reagents, or resolved using conventional techniques (e.g., separated on chiral SFC or HPLC chromatography columns, such as CHIRALPAK® and CHIRALCEL® columns available from DAICEL Corp, or other equivalent columns, using the appropriate solvent or mixture of solvents to achieve suitable separation).

Compounds, e.g., compounds disclosed herein, can be isolated in optically active or racemic forms. Optically active forms may be prepared by resolution of racemic forms or by synthesis from optically active starting materials. All processes used to prepare compounds and intermediates disclosed herein are considered to be part of the present disclosure. When enantiomeric or diastereomeric products are prepared, they may be separated by conventional methods, for example, by chromatography or fractional crystallization.

As used herein, the term “salt” refers to any and all salt forms that compounds disclosed herein can be prepared as, and encompasses pharmaceutically acceptable salts. Pharmaceutically acceptable salts are preferred. However, other salts may be useful, e.g., in isolation or purification steps which may be employed during preparation, and thus, are contemplated to be within the scope of the present disclosure. In general, salts of a compound described herein will be those that provide a composition suitable for administration to a human or animal subject via any suitable route of administration of a pharmaceutical composition.

The term “pharmaceutical composition” as used herein, denotes a composition in which at least one therapeutic or diagnostic agent (e.g., an inhibitor, a viral vector) retains, or partially retains, its intended biological activity or functional form, and in which only pharmaceutically acceptable components are included.

The phrase “pharmaceutically acceptable” means that the substance or composition the phrase modifies must be, within the scope of sound medical judgment, suitable for use in contact with the tissues of humans and lower animals without undue toxicity, irritation, allergic response and the like, and are commensurate with a reasonable benefit/risk ratio. If a substance is part of a composition or formulation, the substance must also be compatible chemically and/or toxicologically with the other ingredients in the composition or formulation.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference, and for example, lists of suitable salts are found in Allen, L. V., Jr., ed., Remington: The Science and Practice of Pharmacy, 22nd Edition, Pharmaceutical Press, London, UK (2012). Pharmaceutically acceptable salts of the compounds described herein include those derived from suitable inorganic and organic acids and inorganic and organic bases.

Pharmaceutically acceptable acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art, such as ion-exchange. Other pharmaceutically acceptable acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Pharmaceutically acceptable base addition salts are formed from inorganic and organic bases. Salts derived from appropriate bases include alkali metal, alkaline earth metal, ammonium, and N⁺ (C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations (e.g., primary, secondary, tertiary, quaternary amine cations), for example, formed using counterions such as halide, hydroxide, carboxylate, sulfate, phosphate, nitrate, lower alkyl sulfonate, and aryl sulfonate. Examples of organic amines from which base addition salts can be derived include, but are not limited to, isopropylamine, benzathine, cholinate, diethanolamine, diethylamine, lysine, meglumine, piperazine and tromethamine.

A salt (e.g., pharmaceutically acceptable salt) of a compound described herein can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

It will be understood that when the compound described herein contains more than one basic moiety or more than one acidic moiety, each such moiety can independently be involved in forming an acid addition salt form or base addition salt form, with all possible salt forms being included in this disclosure. Further, when two or more moieties of a compound are in salt form, the anions or cations forming the two or more salt forms can be the same or different. Typically, the anions or cations forming the two or more salt forms are the same. Typical molar ratios of an anion or cation in a salt of a compound of the present disclosure to a compound described herein are 3:1, 2:1, 1:1, 2:1, 3:1, 4:1 and 5:1. In some embodiments, the molar ratio of an anion or cation (e.g., anion) in a salt of a compound described herein to the compound is 1:1.

Lists of suitable salts are found in Allen, L. V., Jr., ed., Remington: The Science and Practice of Pharmacy, 22nd Edition, Pharmaceutical Press, London, UK (2012), the relevant disclosure of which is hereby incorporated by reference in its entirety.

Compounds described herein are also provided, and can be administered, as a free base.

The term “solvate” means a physical association of a compound of the present disclosure with one or more solvent molecules, whether organic or inorganic. This physical association includes hydrogen bonding. In certain instances, the solvate will be capable of isolation, for example, when one or more solvent molecules are incorporated in the crystal lattice of a crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution phase and solid phase solvates. Examples of solvates include, but are not limited to, hydrates, ethanolates, methanolates, and isopropanolates. Methods of solvation are generally known in the art.

A “pharmaceutically acceptable carrier” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like, and combinations thereof, as would be known to those skilled in the art (see, for example, Allen, L. V., Jr. et al., Remington: The Science and Practice of Pharmacy (2 Volumes), 22nd Edition, Pharmaceutical Press (2012).

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

As used herein, a subject (e.g., a human) is “in need of” a treatment if such subject would benefit biologically, medically or in quality of life from such treatment.

The term “administer,” “administering,” or “administration” refers to implanting, absorbing, ingesting, injecting, inhaling, or otherwise introducing the referenced material (e.g., compound described herein, or a pharmaceutically acceptable salt thereof, or a composition thereof), in or on a subject.

The terms “treatment,” “treat,” and “treating” refer to administration of a medication or medical care to a subject, such as a human, having a disease or condition of interest, e.g., a cancer, and includes: (i) preventing the disease or condition from occurring in a subject, in particular, when such subject is predisposed to the condition but has not yet been diagnosed as having it; (ii) inhibiting the disease or condition, e.g., arresting its development; (iii) relieving the disease or condition, e.g., causing regression of the disease or condition; and/or (iv) relieving the symptoms resulting from the disease or condition (e.g., pain, weight loss, cough, fatigue, weakness, etc.). Treating thus includes reversing, alleviating, delaying the onset of, and/or inhibiting the progress of a disease (e.g., a disease described herein). In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms. Treatment may also be continued after symptoms have resolved, for example, to delay or prevent recurrence.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. Alternatively, an effective amount is a prophylactically effective amount. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition, for example, an amount sufficient to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating in any disease or condition described.

A “prophylactically effective amount” of a compound described herein is an amount sufficient to prevent a condition, or one or more symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, “inhibition”, “inhibiting”, “inhibit” and “inhibitor”, and the like, refer to the ability of a compound to reduce, slow, halt, or prevent the activity of a biological process (e.g., the activity of an activin receptor-like kinase (e.g., ALK-5) in a subject or cell) or change thereby the progress of a disease by, for example, altering a signaling pathway, for example, altering TGF-β1 signaling.

As will be appreciated by one of skill in the art, reference herein to “compounds of the disclosure,” “compounds described herein,” and the like refers to any compound discussed herein (e.g., a host protein inhibitor), as well as isomers, such as stereoisomers (including diastereoisomers, enantiomers and racemates), geometrical isomers, conformational isomers (including rotamers and astropisomers), tautomers, isotopically labeled compounds (including deuterium substitutions), and inherently formed moieties (e.g., polymorphs and/or solvates, such as hydrates) thereof. When a moiety is present that is capable of forming a salt, then salts are included as well, in particular, pharmaceutically acceptable salts. Compounds of the present disclosure can also be provided as amorphous solids or crystalline solids. Lyophilization can be employed to provide the compounds of the present disclosure as a solid. Such solid forms are also included in these terms. For example, a description using the structural representation of a free base form of a compound of the disclosure contemplates hydrates, solvates, polymorphs, co-crystals, salts, tautomers, stereoisomers, and isotopically labeled derivatives of the compounds. For example, a structural representation of a free base form of a compound of the disclosure contemplates all salt forms (e.g., pharmaceutically acceptable salt forms) of the compound. For example, a structural representation lacking stereochemical designation of a compound of the disclosure having asymmetric carbon centers contemplates all isomers, including isolation of one or more particular isomers in all levels of enantiomeric or diastereomeric purity. For example, a structural representation of a compound of the disclosure having keto/enol tautomeric forms in one particular tautomeric form contemplates all tautomeric forms of the compound.

As used herein, “tautomers” refers to compounds whose structures differ markedly in the arrangement of atoms, but which exist in easy and rapid equilibrium. It is to be understood that compounds of the present disclosure may be depicted as different tautomers. It should also be understood that when compounds bave tautomeric forms, all tautomeric forms are intended to be within the scope of the disclosure, and the naming of the compounds does not exclude any tautomeric form. A tautomer is one of two or more structural isomers that exist in equilibrium and are readily converted from one isomeric form to another. This reaction results in the formal migration of a hydrogen atom accompanied by a shift of adjacent conjugated double bonds. In solutions where tautomerization is possible, a chemical equilibrium of the tautomers can be reached. The exact ratio of the tautomers depends on several factors, including temperature, solvent, and pH. The concept of tautomers that are interconvertable by tautomerizations is called tautomerism.

As used herein, an “amino acid” or “residue” refers to any naturally or non-naturally occurring amino acid, any amino acid derivative or any amino acid mimic known in the art. Included are the L- as well as the D-forms of the respective amino acids, although the L-forms are usually preferred. In some embodiments, the term relates to any one of the 20 naturally occurring amino acids: glycine (Gly), alanine (Ala), valine (Val), leucine (Leu), isoleucine (Ile), proline (Pro), cysteine (Cys), methionine (Met), serine (Ser), threonine (Thr), glutamine (Gin), asparagine (Asn), glutamic acid (Glu), aspartic acid (Asp), lysine (Lys), histidine (His), arginine (Arg), phenylalanine (Phe), tryptophan (Trp), and tyrosine (Tyr) in their L-form. In certain embodiments, the amino acid side-chain may be a side-chain of Gly, Ala, Val, Leu, Ile, Met, Cys, Ser, Thr, Trp, Phe, Lys, Arg, His, Tyr, Asn, Gln, Asp, Glu, or Pro.

As used herein, except where the context requires otherwise, the term “comprise” and variations of the term, such as “comprising”, “comprises” and “comprised”, are not intended to exclude further additives, components, integers or steps. The terms “including” and “comprising” may be used interchangeably. As used herein, the phrases “selected from the group consisting of”, “chosen from”, and the like, include mixtures of the specified materials. Where a numerical limit or range is stated herein, the endpoints are included. Also, all values and subranges within a numerical limit or range are specifically included as if explicitly written out. References to an element in the singular is not intended to mean “one and only one” unless specifically stated, but rather “one or more.” Unless specifically stated otherwise, terms such as “some” refer to one or more, and singular terms such as “a”, “an” and “the” refer to one or more.

The term “oligopeptide” is used to refer to a peptide with fewer members of amino acids as opposed to a polypeptide or protein. Oligopeptides described herein, are typically comprised of about two to about forty amino acid residues. Oligopeptides include dipeptides (two amino acids), tripeptides (three amino acids), tetrapeptides (four amino acids), pentapeptides (five amino acids), hexapeptides (six amino acids), heptapeptides (seven amino acids), octapeptides (eight amino acids), nonapeptides (nine amino acids), decapeptides (ten amino acids), undecapeptides (eleven amino acids), dodecapeptides (twelve amino acids), icosapeptides (twenty amino acids), tricontapeptides (thirty amino acids), tetracontapeptides (forty amino acids), etc. Oligopeptides may also be classified according to molecular structure: aeruginosas, cyanopeptolins, microcystins, microviridins, microginins, anabaenopeptins and cyclamides, etc. Homo-oligopeptides are oligopeptides comprising the same amino acid. In preferred embodiments, homo-oligopeptides comprise 10 amino acid poly-valine, poly-alanine, and poly-glycine hexamers.

The meaning of the term “peptides” are defined as small proteins of two or more amino acids linked by the carboxyl group of one to the amino group of another. Accordingly, at its basic level, peptide synthesis of whatever type comprises the repeated steps of adding amino acid or peptide molecules to one another or to an existing peptide chain. The term “peptide” generally has from about 2 to about 100 amino acids, whereas a polypeptide or protein has about 100 or more amino acids, up to a full length sequence which may be translated from a gene. Additionally, as used herein, a peptide can be a subsequence or a portion of a polypeptide or protein. In certain embodiments, the peptide consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues. In preferred embodiments, the peptide is from between about 30 to about 100 amino acids in length. In some embodiments, the peptide is from between about 40 to about 100 amino acids in length.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the term “prodrug” is intended to encompass therapeutic biologics which, under physiologic conditions, are converted into the therapeutically active biologics of the present disclosure. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present disclosure. In certain embodiments, some or all of the molecules in a composition represented above can be replaced with the corresponding suitable prodrug, e.g., wherein a hydroxyl in the parent molecule is presented as an ester or a carbonate or carboxylic acid present in the parent therapeutic biologic is presented as an ester.

The meaning of the term “protein” is defined as a linear polymer built from about 20 different amino acids. The type and the sequence of amino acids in a protein are specified by the DNA that produces them. In certain embodiments, the sequences can be natural and unnatural. The sequence of amino acids determines the overall structure and function of a protein. In some embodiments, proteins can contain 50 or more residues. In preferred embodiments, proteins can contain greater than about 101 residues in length. A protein's net charge can be determined by two factors: 1) the total count of acidic amino acids vs. basic amino acids; and 2) the specific solvent pH surroundings, which expose positive or negative residues. As used herein, “net positively or net negatively charged proteins” are proteins that, under non-denaturing pH surroundings, have a net positive or net negative electric charge. In general, those skilled in the art will recognize that all proteins may be considered “net negatively charged proteins”, regardless of their amino acid composition, depending on their pH and/or solvent surroundings. For example, different solvents can expose negative or positive side chains depending on the solvent pH. Proteins or peptides are preferably selected from any type of enzyme or antibodies or fragments thereof showing substantially the same activity as the corresponding enzyme or antibody. Proteins or peptides may serve as a structural material (e.g. keratin), as enzymes, as hormones, as transporters (e.g. hemoglobin), as antibodies, or as regulators of gene expression. Proteins or peptides are required for the structure, function, and regulation of cells, tissues, and organs.

The term “substantially” as used herein, refers to a majority of, or mostly, as in at least about 50%, 60%, 70%, 80%, 90%, 95%, 96%, 97%, 98%, 99%, 99.5%, 99.9%, 99.99%, or at least about 99.999% or more.

It is understood that the specific order or hierarchy of steps in the methods or processes disclosed is an illustration of exemplary approaches. Based upon design preferences, it is understood that the specific order or hierarchy of steps in the methods or processes may be rearranged. Some of the steps may be performed simultaneously. The accompanying methods claims present elements of the various steps in a sample order, and are not meant to be limited to a specific hierarchy or order presented. A phrase such as “embodiment” does not imply that such embodiment applies to all configurations of the subject technology. A disclosure relating to an embodiment may apply to all embodiments, or one or more embodiments. A phrase such as an embodiment may refer to one or more embodiments and vice-versa.

Unless otherwise defined, all terms of art, notations and other scientific terminology used herein, are intended to have the meanings commonly understood by those of skill in the art to which this disclosure pertains. In some cases, terms with commonly understood meanings are defined herein, for clarity and/or for ready reference, and the inclusion of such definitions herein, should not necessarily be construed to represent a substantial difference over what is generally understood in the art. The techniques and procedures described or referenced herein, are generally well understood and commonly employed using conventional methodology by those skilled in the art. As appropriate, procedures involving the use of commercially available kits and reagents are generally carried out in accordance with manufacturer defined protocols and/or parameters unless otherwise noted.

### EXAMPLES

### Example 1. Identification of Host Proteins

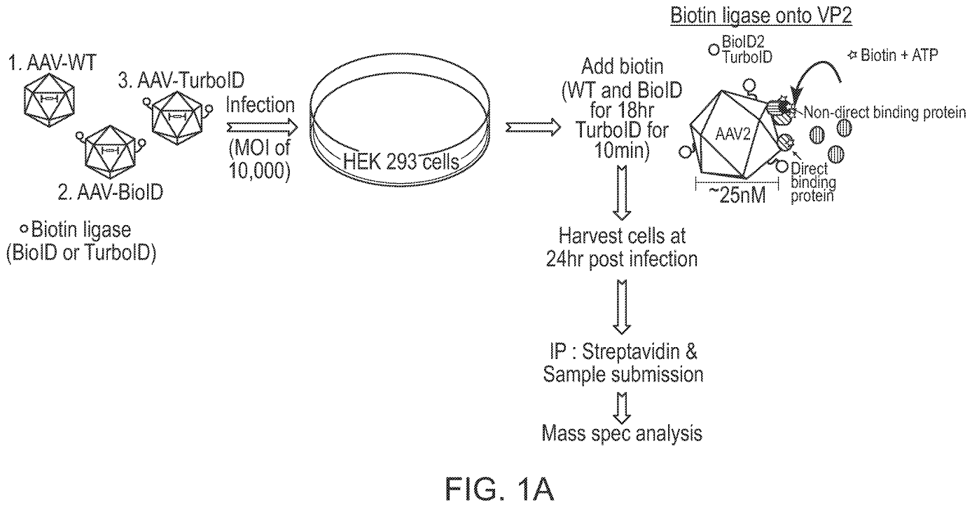

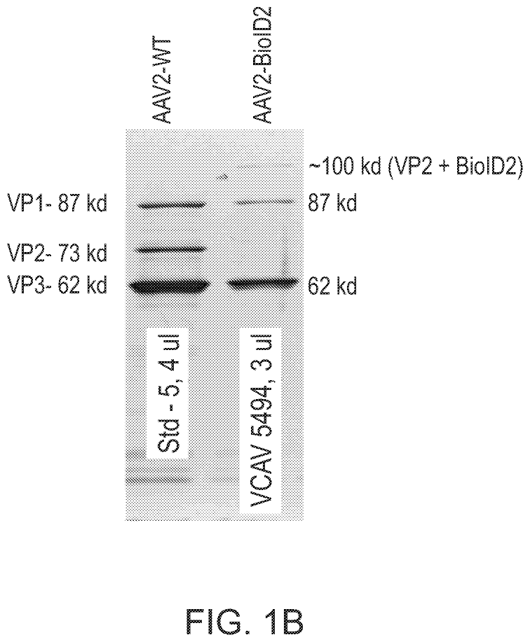

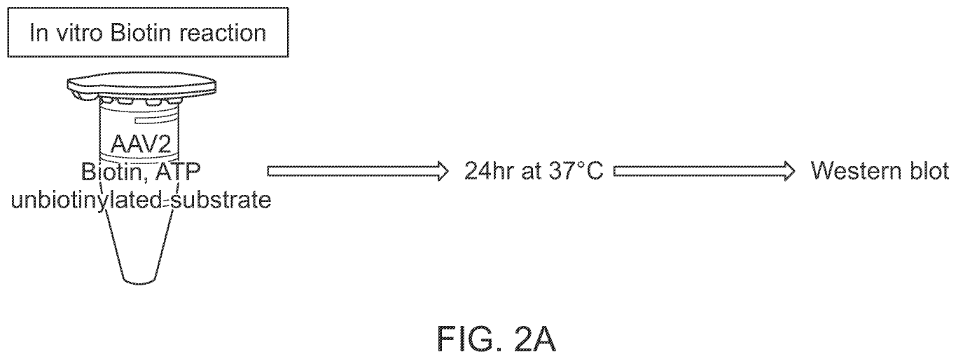

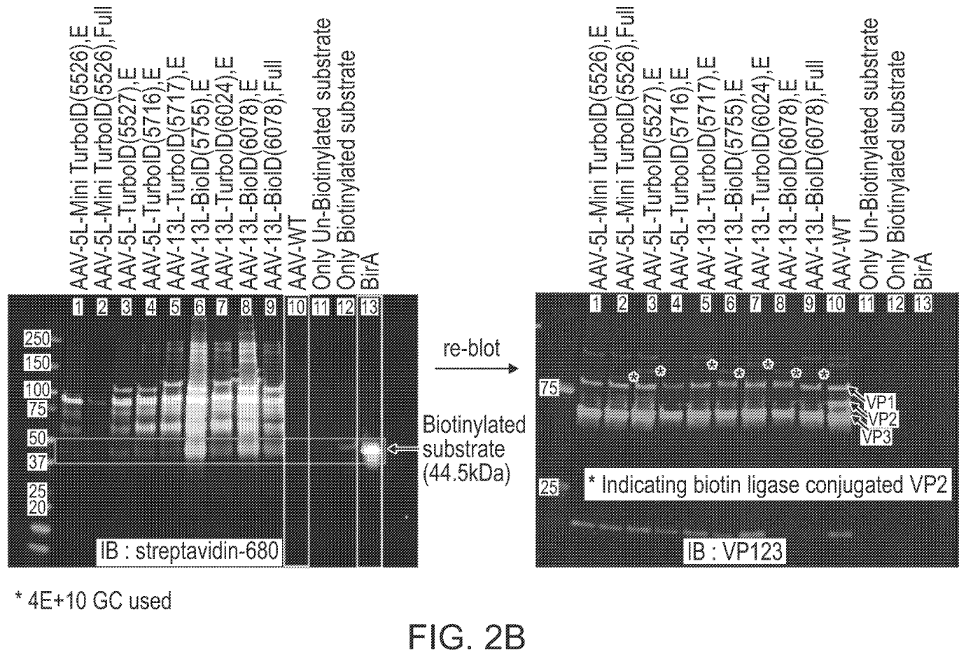

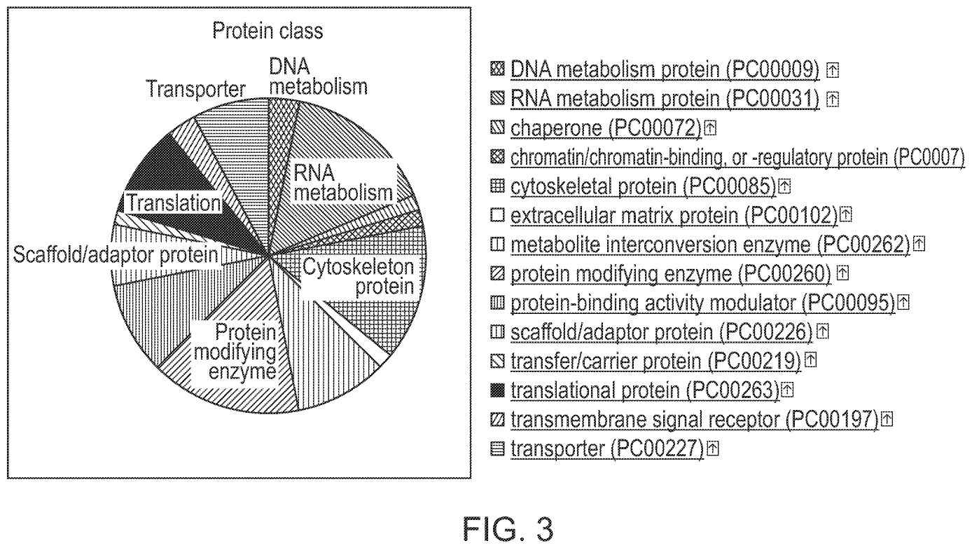

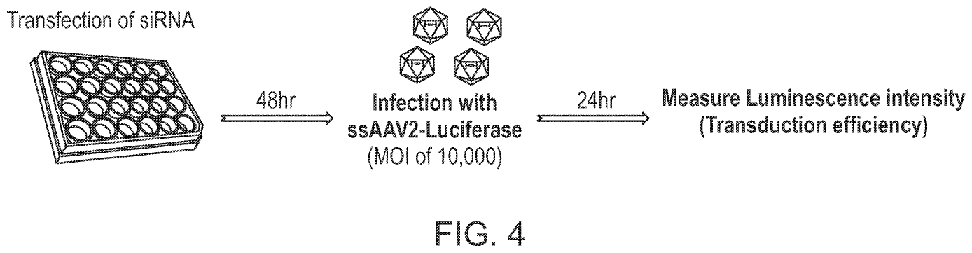

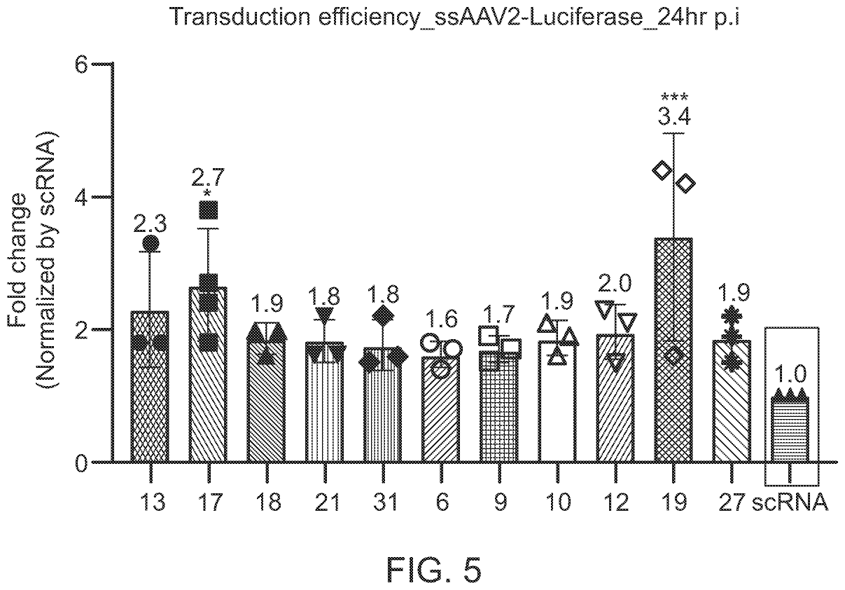

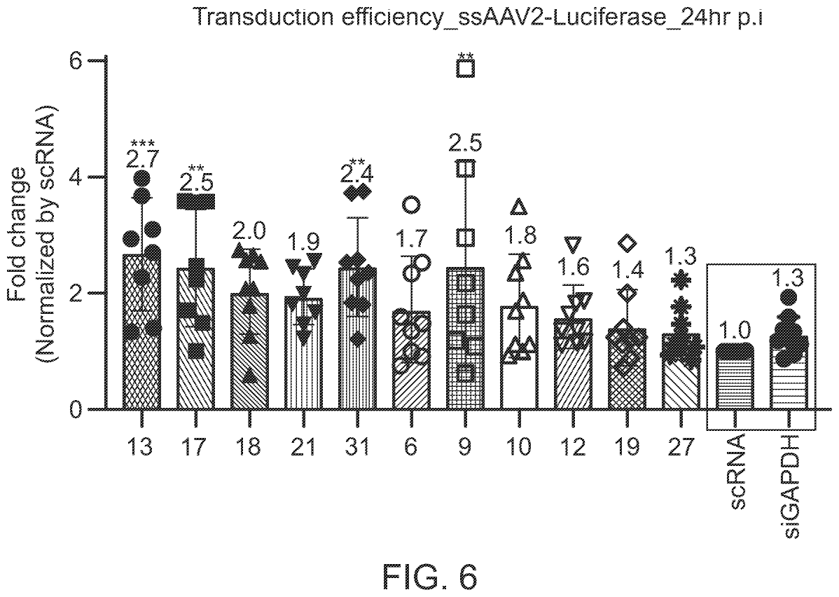

Host proteins involved in AAV transduction were identified by proximity labeling using proximity-dependent biotin identification (BioID), a method that allows the identification of proteins in the close vicinity of a protein of interest in living cells (see Varnaitė, R. and MacNeill, S. A., 2016. Proteomics, 16(19), pp. 2503-2518, which is incorporated herein by reference in its entirety). BioID relies on fusion of the protein of interest with a mutant form of the biotin ligase enzyme BirA (BirA*) that is capable of promiscuously biotinylating proximal proteins irrespective of whether these interact directly or indirectly with the fusion protein or are merely located in the same subcellular neighborhood. The covalent addition of biotin allows the labeled proteins to be purified from cell extracts on the basis of their affinity for streptavidin and identified by mass spectrometry, as illustrated in FIGS. 1A and 1B. Western blotting was used to separate and identify proteins by molecular weight, as shown in FIGS. 2A and 2B. 77ea host proteins were identified as candidates involved in AAV transduction by mass spectroscopy analysis. Gene ontology enrichment analysis was performed with 77 host protein candidates (FIG. 3) to identify gene function. Out of the 77 host protein candidates, 11 candidates were tested for transduction efficiency in HEK 293 cells (FIGS. 4 and 5) and HeLA cells (FIG. 6). Six host proteins (see Table 1) were then selected for further evaluation.

### Example 2. Transduction Efficiency in Other AAV Serotypes

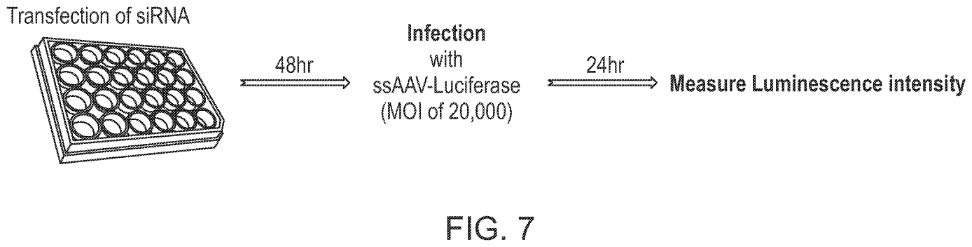

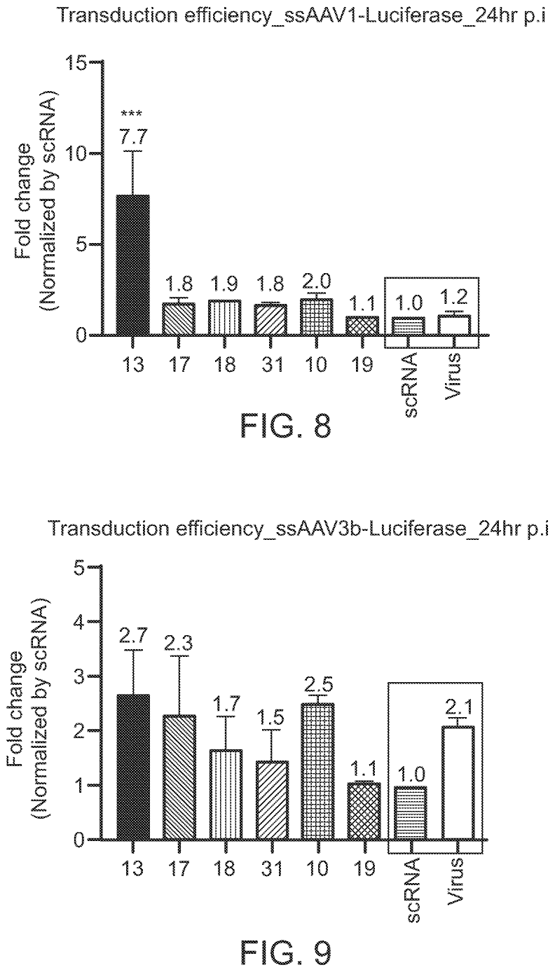

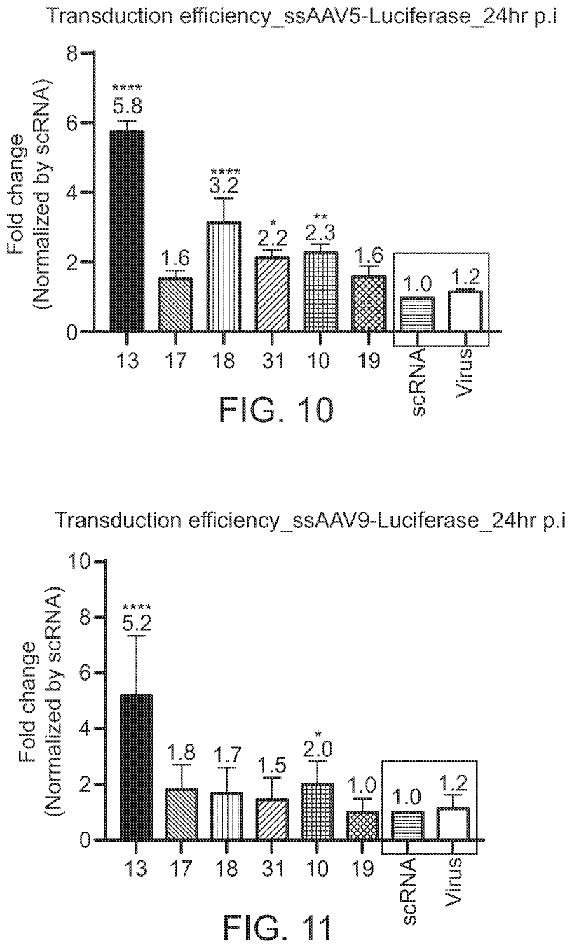

Transduction efficiency in other AAV serotypes was evaluated based on an example protocol illustrated in FIG. 7. AAV serotypes and tissue tropism in human are presented in https_biovian_com/news/beginners-guide-to-the-production-of-aav-vectors-for-gene-therapy-mentored-by-a-cdmo/, which is incorporated herein by reference in its entirety. FIGS. 8-11 show the transduction efficiency results. Knock-down of No. 13 (eIF4A3) increased AAV1, 5 and 9 transduction but not AAV3b.

### Example 3. Knock-Down of Host Protein and its Influence on AAV Vector Transduction

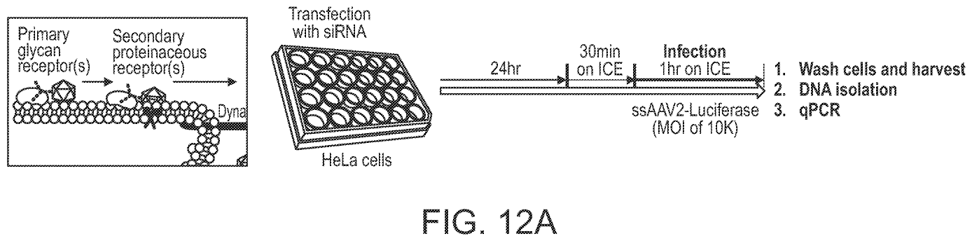

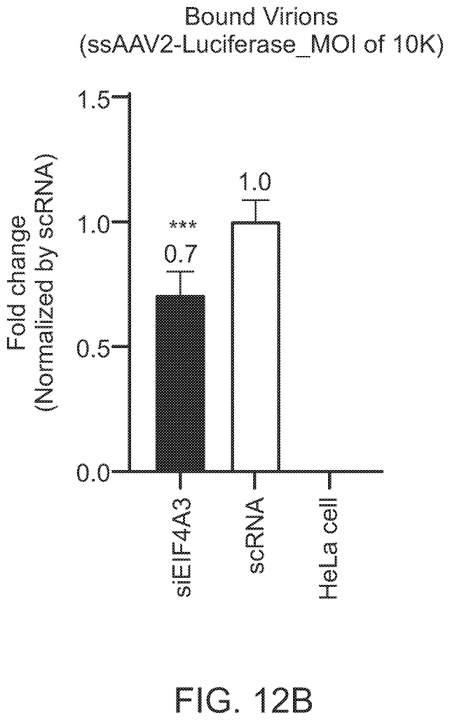

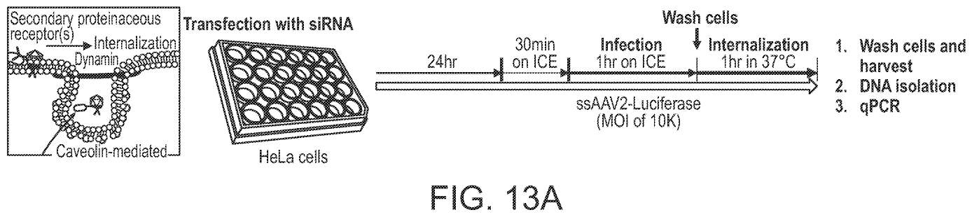

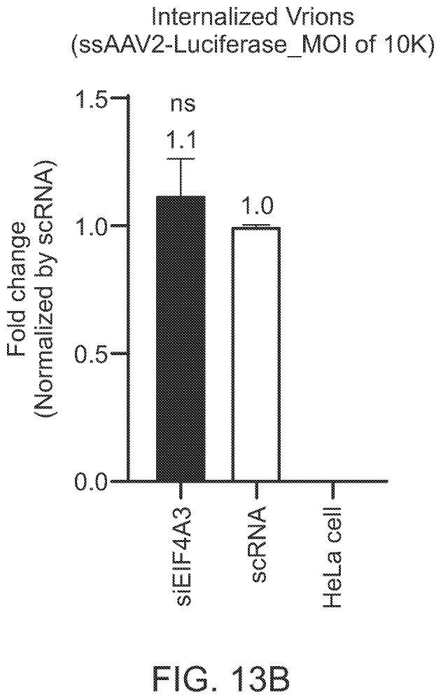

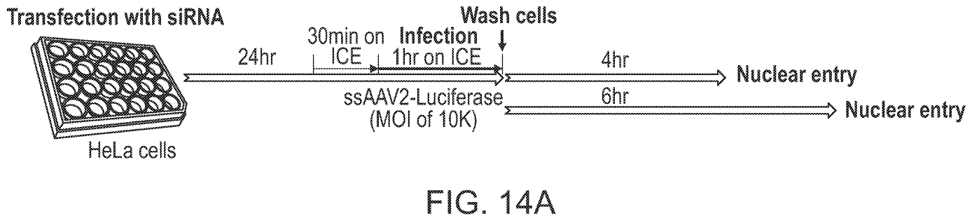

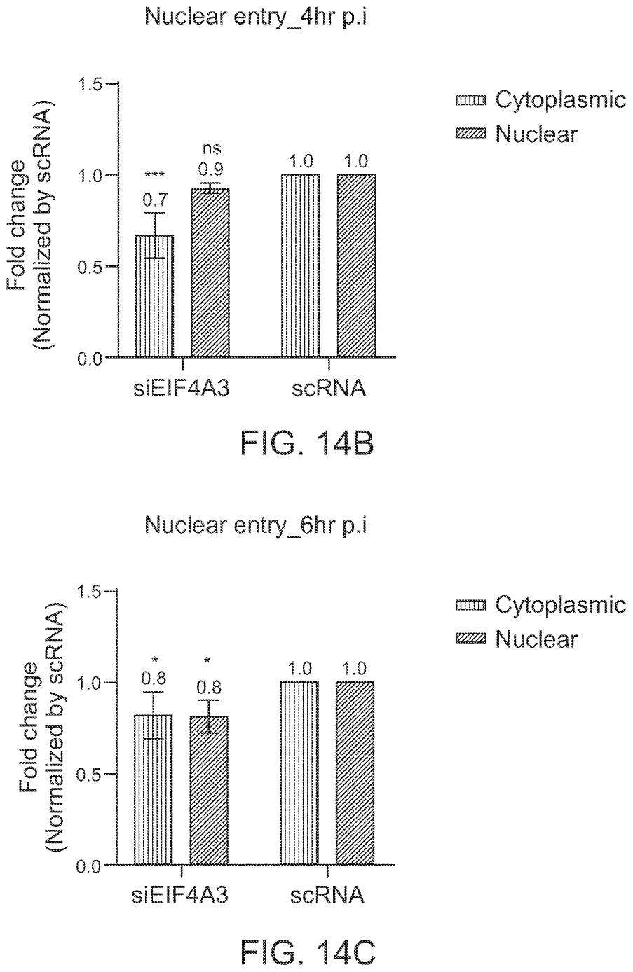

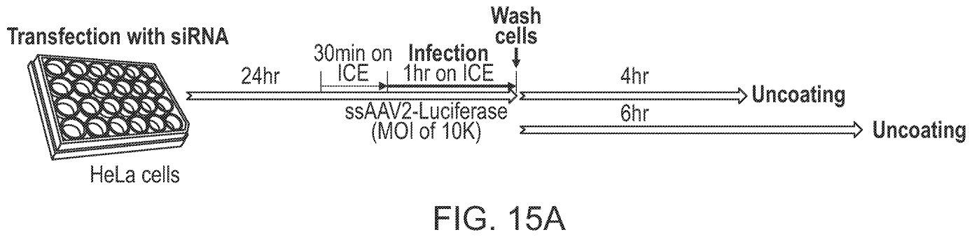

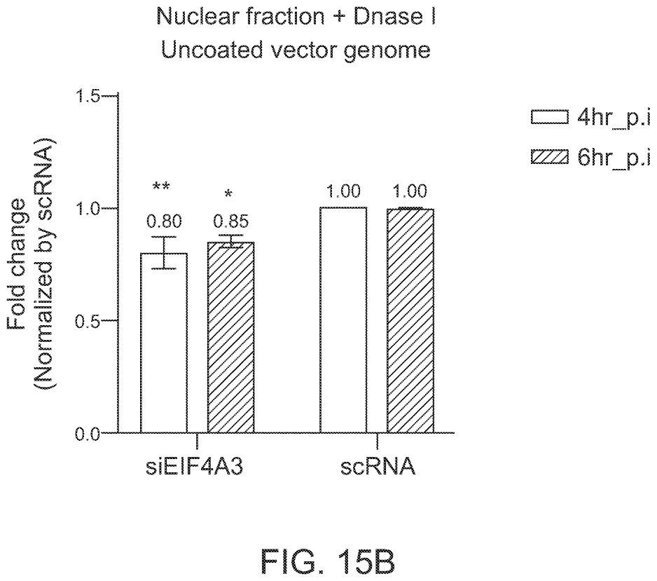

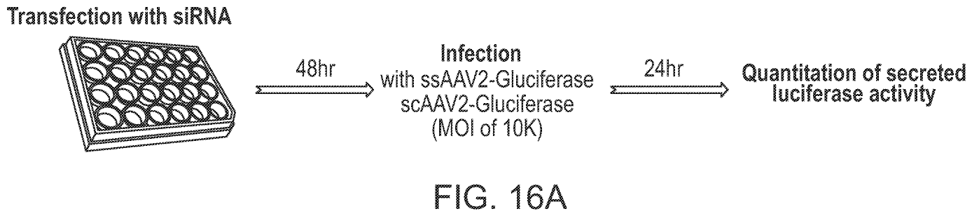

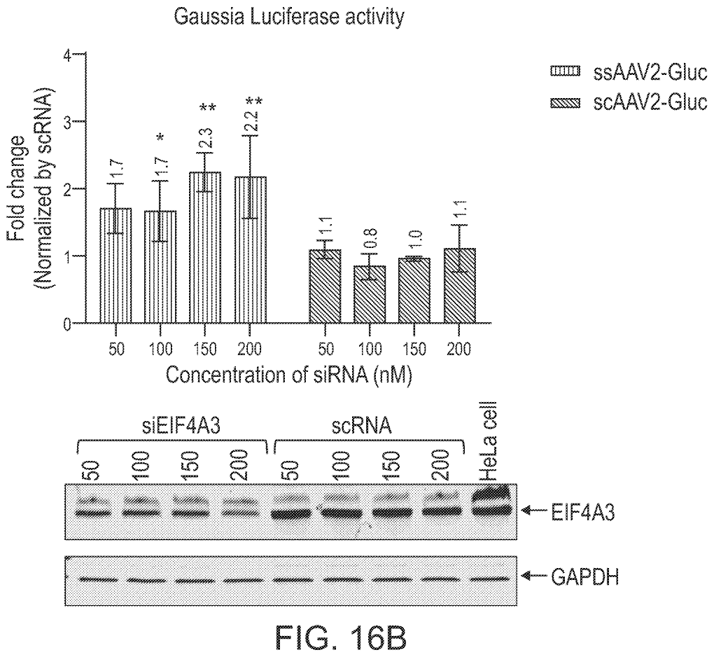

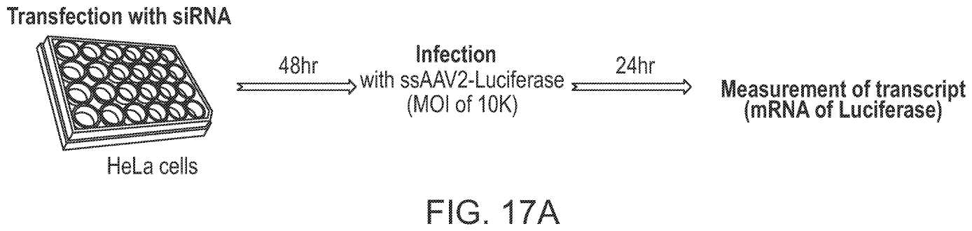

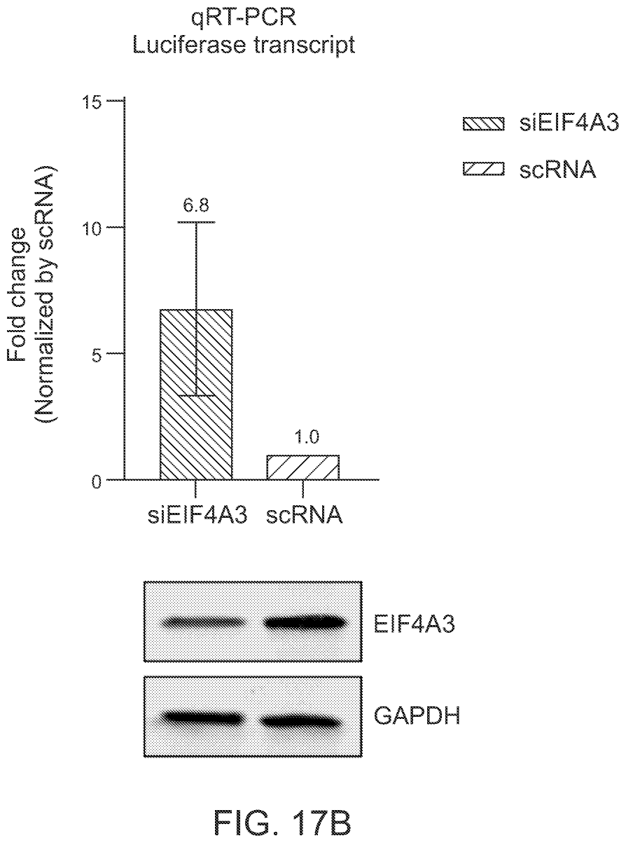

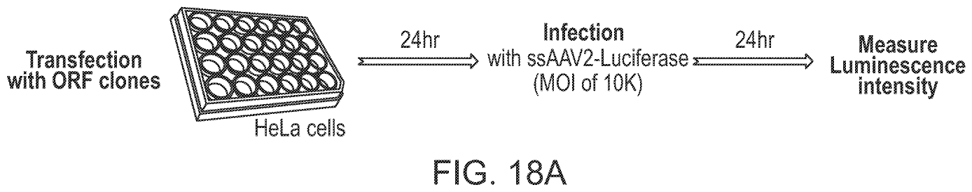

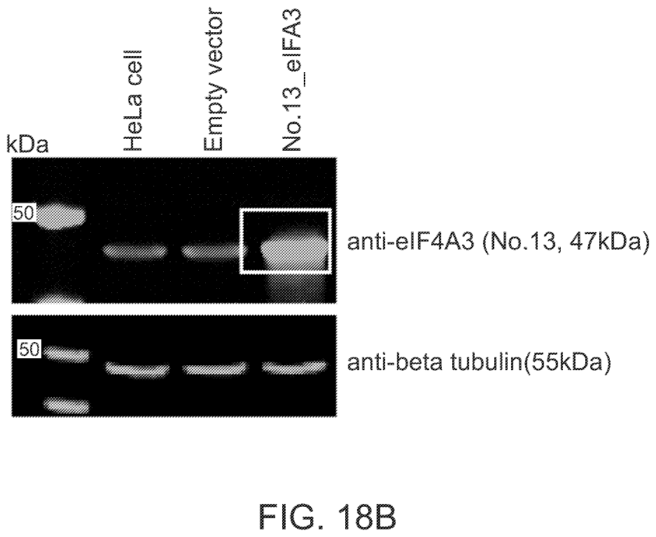

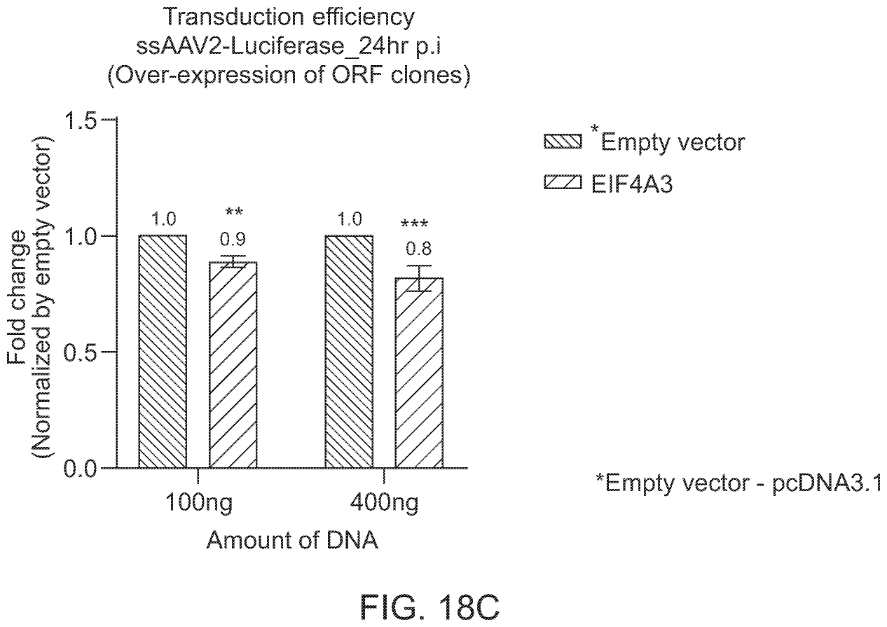

The influence of host protein (e.g., eIF4A3) knock-down on multiple stages of AAV vector transduction was examined based on the schemes in FIGS. 12A, 13A, 14A, 15A, 16A. 17A and 18A. As shown in FIG. 12B, knock-down of eIF4A3 indicates a reduction of AAV attachment to the cell membrane. As shown in FIG. 13B, knock-down of eIF4A3 indicates that eIF4A3 does not play a role in cellular entry of AAV internalization. As shown in FIGS. 14B and 14C, knock-down of eIF4A3 decreased nuclear entry of AAV. As shown in FIG. 15B, knock-down of eIF4A3 decreased uncoated vector genome. As shown in FIG. 15B, knock-down of eIF4A3 decreased uncoated vector genome. As shown in FIG. 16B, knock-down of eIF4A3 affects the conversion of single strand to double strand DNA. As shown in FIG. 17B, knock-down of eIF4A3 increased AAV genome transcription. As shown in FIGS. 18B and 18C, overexpression of open reading frame (ORF) clones slightly reduced AAV transduction.

### Example 4. Host Protein Inhibitor on AAV Transduction

eIF4A3 consists of 411 amino acids: 1. Q motif at positions 38-60; 2. DEAD (Asp-Glu-Ala-Asp) box motif at positions 187-190; 3. ATP-dependent helicase motif at amino acids 69-239 and a C-terminal helicase motif at amino acids 250-411 (pre-mRNA processing) (see Huang Z. et al., Front Oncol. 2021; 11:714665, which is incorporated herein by reference in its entirety). A eIF4A inhibitor, 3~[4-[(3R)-4-(4-bromobenzoyl)-3-(4-chlorophenyl) piperazine-1-carbonyl]-5-methylpyrazol-1-yl]benzonitrile (eIF4A3-IN-1), was investigated on its influence on AAV transduction. eIF4A3-IN-1 binds to a non-ATP binding site of eIF4A3 and shows significant cellular nonsense-mediated RNA decay (NMD) inhibition.

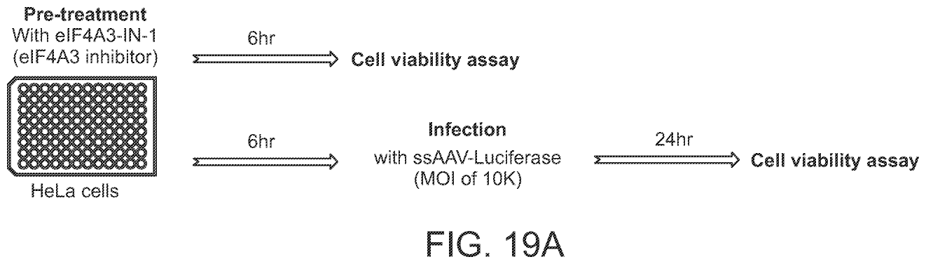

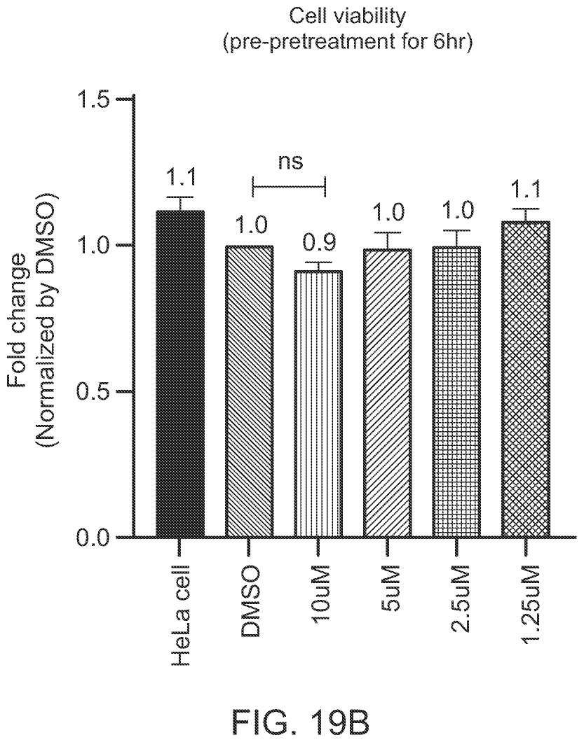

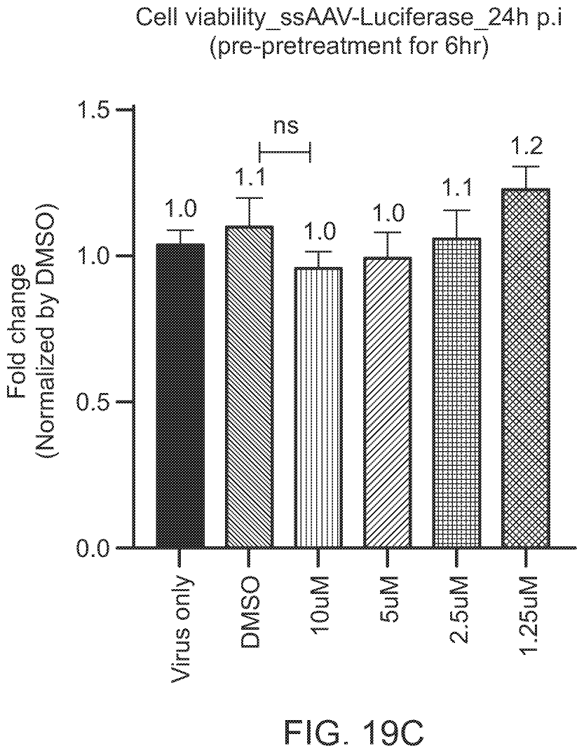

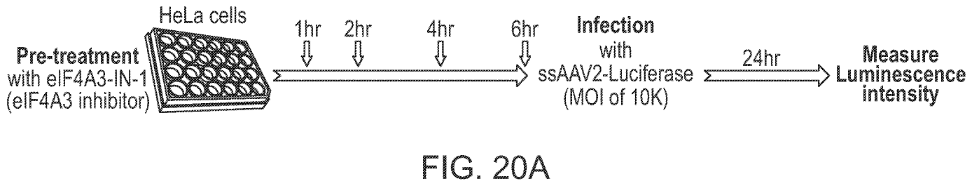

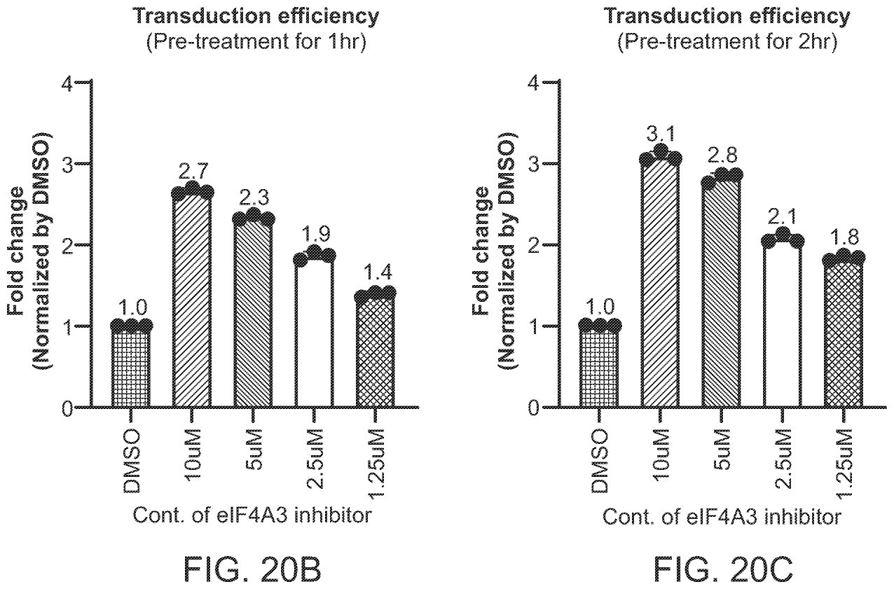

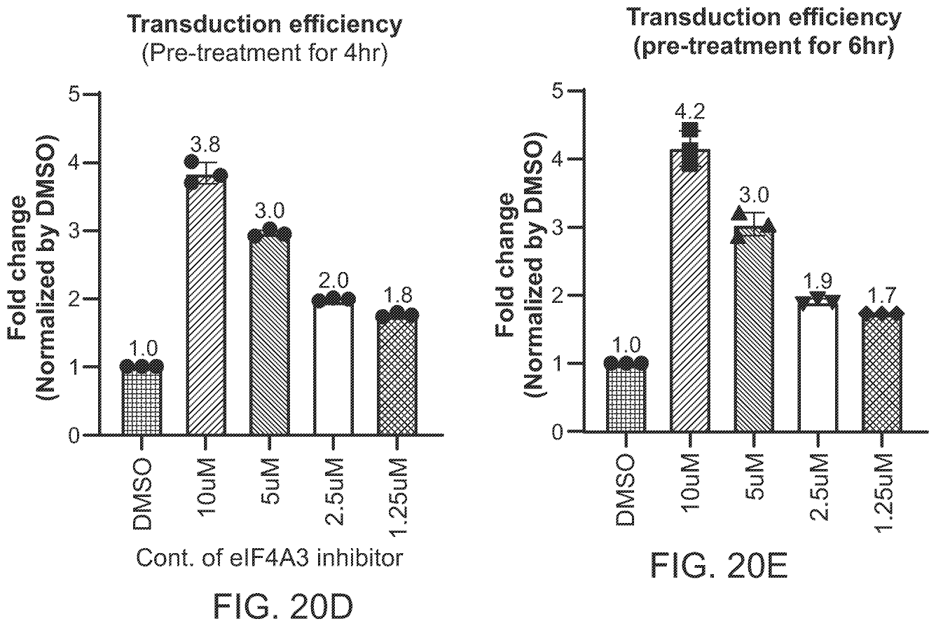

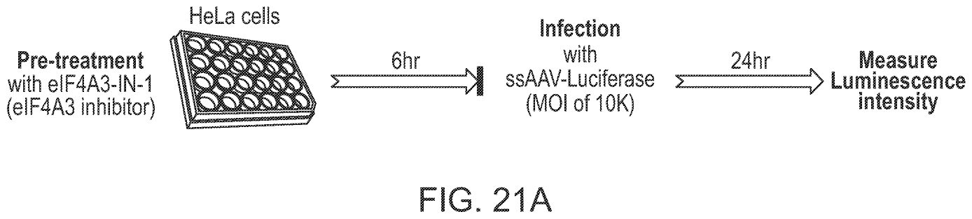

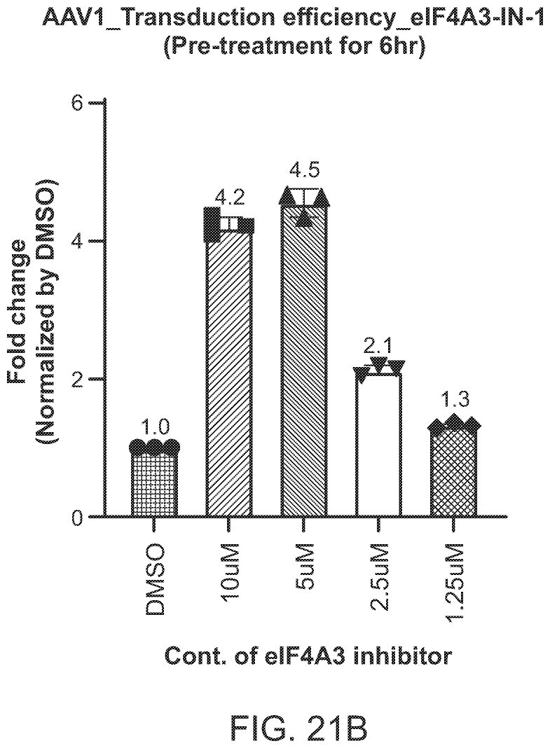

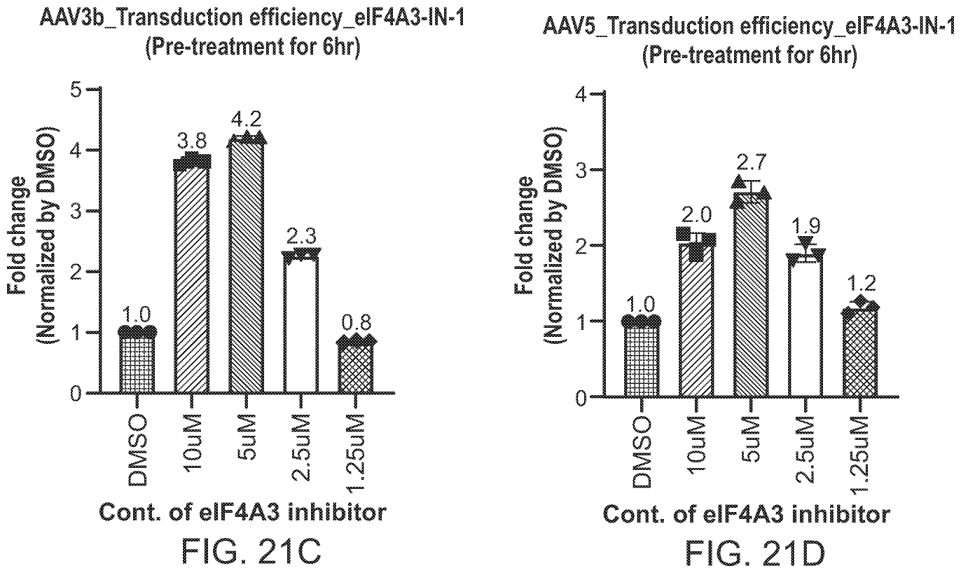

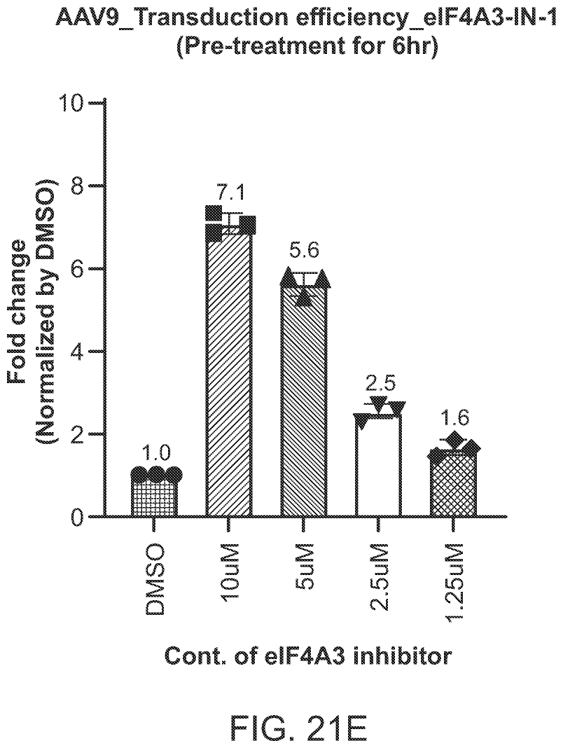

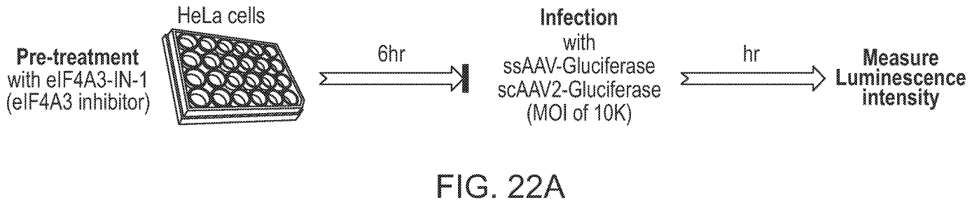

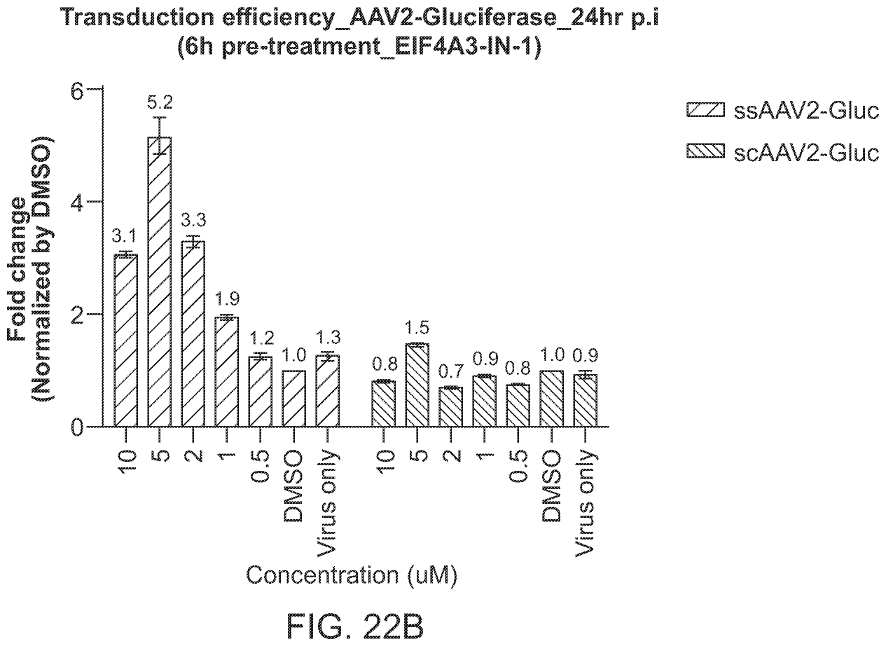

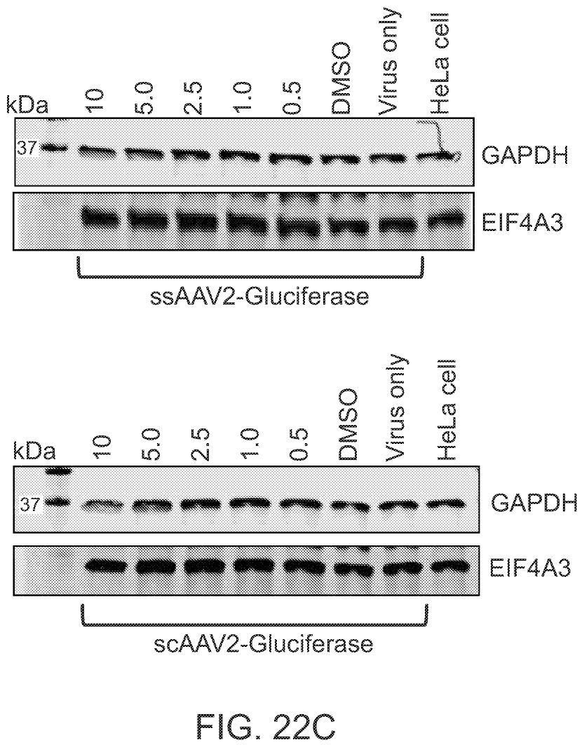

Based on the cell viability protocol illustrated in FIG. 19A, eIF4A3-IN-1 was found to not affect cell viability (FIGS. 19B and 19C). eIF4A3-IN-1 was found to increase AAV transduction in a dose and time-dependent manner, following pre-treatment of HeLa cells with eIF4A3-IN-1 for various time periods before infection with ssAAV2-Luciferase (FIGS. 20A-E). In addition to AAV2, eIF4A3-IN-1 also increases AAV transduction in other AAV stereotypes (FIGS. 21A-E)-AAV1, AAV3b, AAV5, and AAV9. eIF4A3-IN-1 increases ssAAV-Gaussia luciferace transduction but not scAAV-Gaussia luciferace (FIGS. 22A-C).

Another eIF4A inhibitor, 4-[(2S,3R,4S,5S,6R)-4-[(dimethylamino)methyl]-2,3-dihydroxy-10,12-dimethoxy-5-phenyl-7-oxa-11-azatricyclo[6.4.0.02,6]dodeca-1(12), 8,10-trien-6-yl]benzonitrile (zotatifin), was investigated on its influence on AAV transduction. Zotatifin promotes eIF4A binding to specific mRNA sequences with recognition motifs in the 5′-UTRs (IC450=2 nM) and interferes with the assembly of the eIF4F initiation complex. Zotatifin shows robust antiviral effects, and effectively reduces viral infectivity by inhibiting SARS-COV-2 NP protein biogenesis. Zotatifin also induces cell apoptosis.

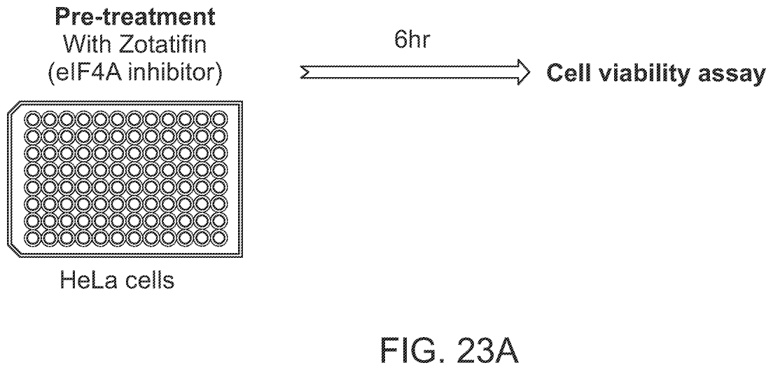

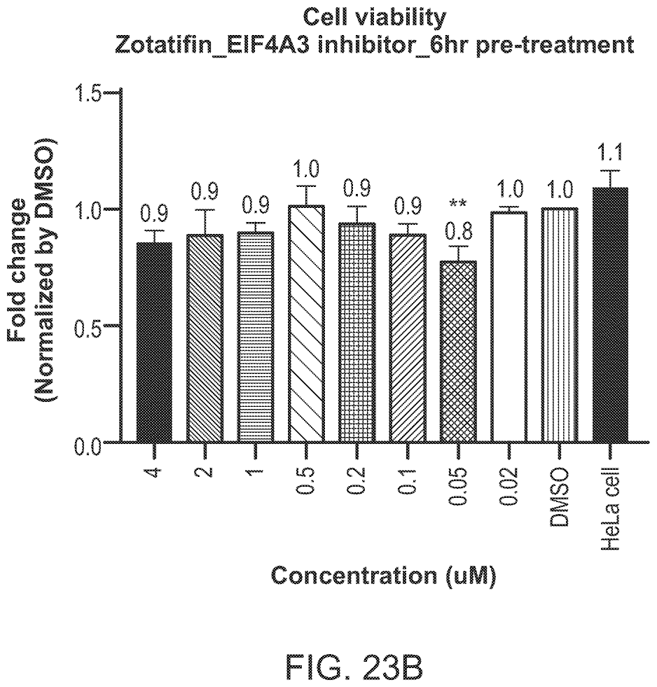

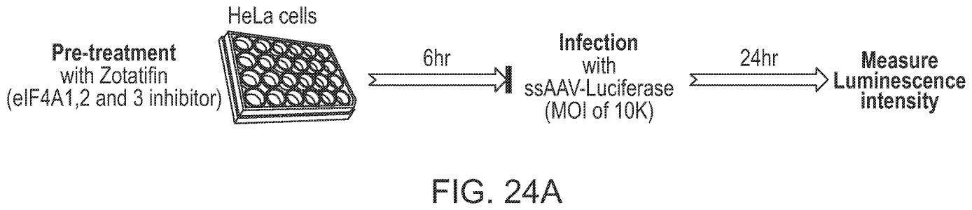

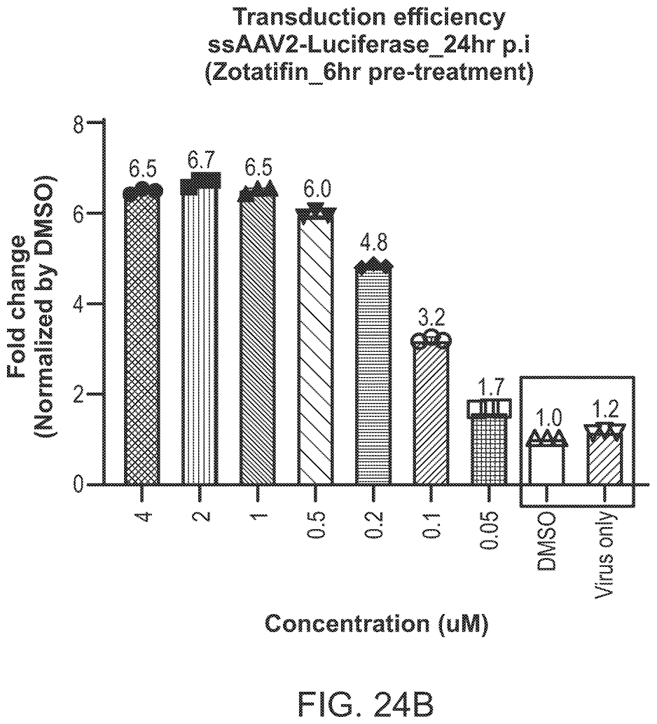

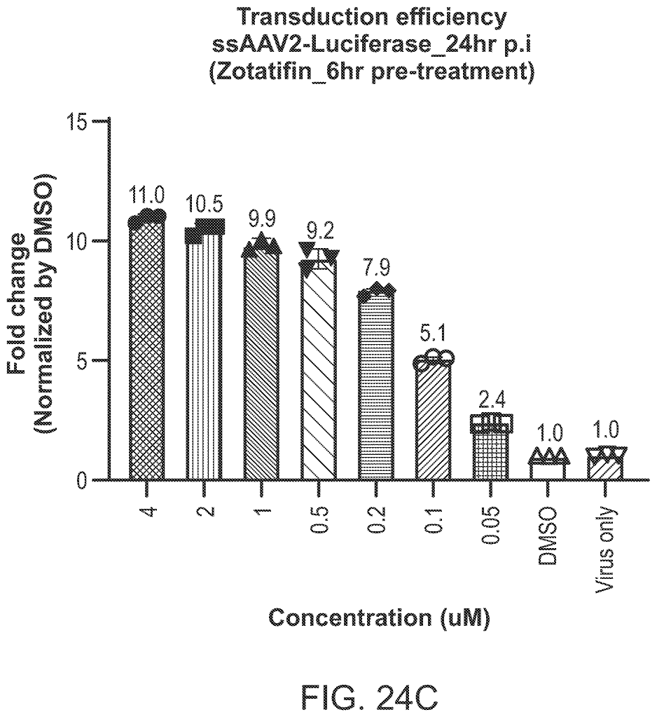

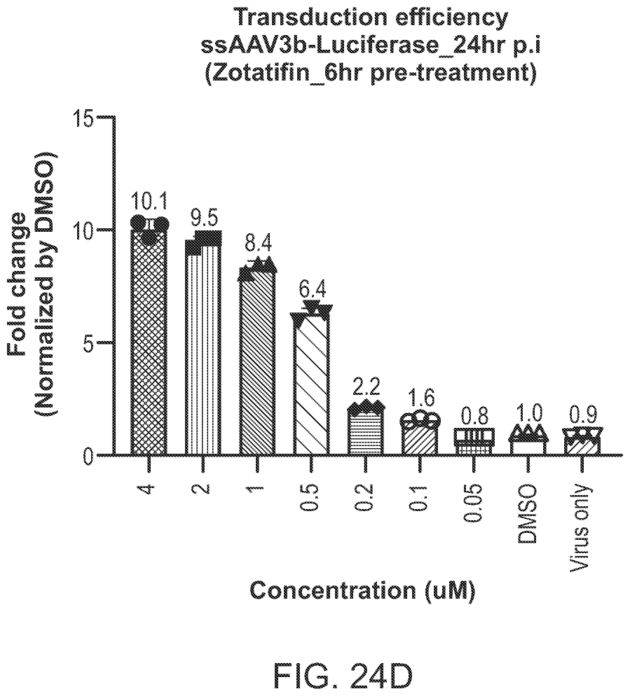

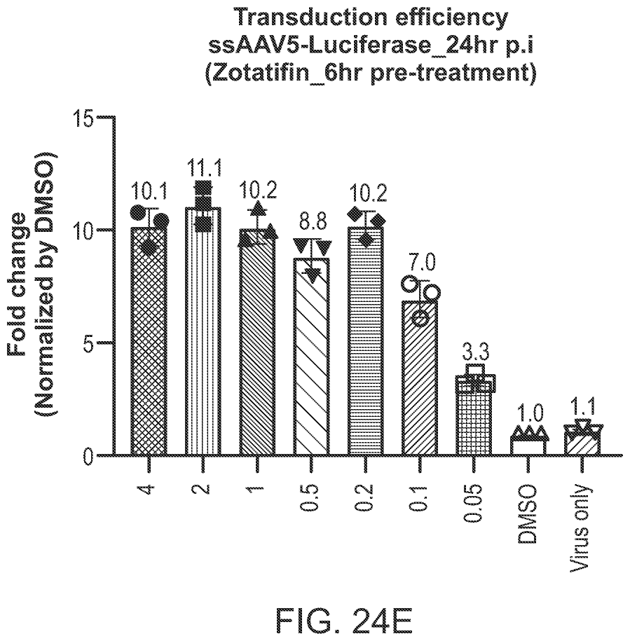

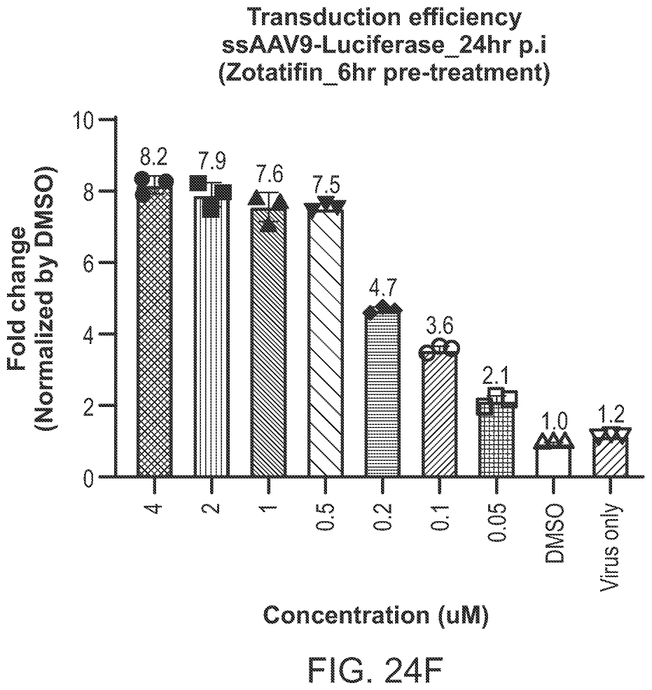

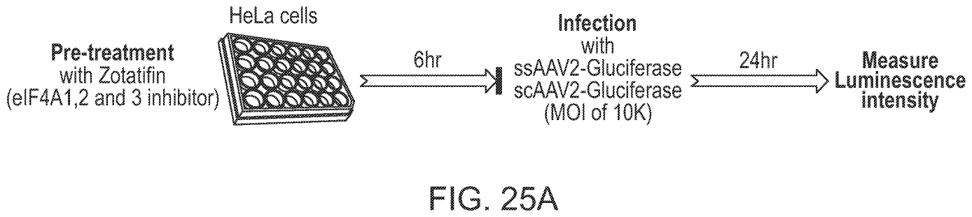

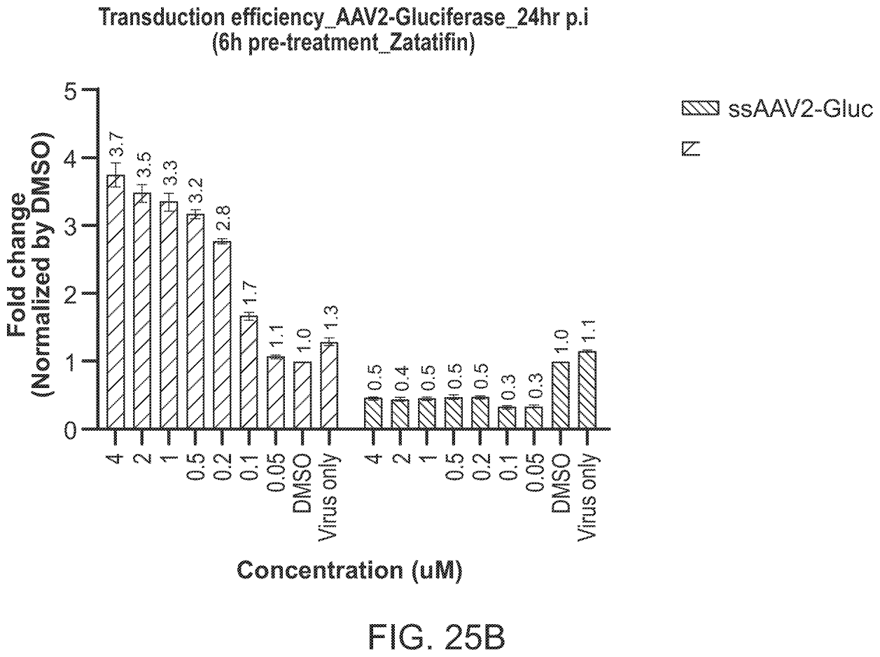

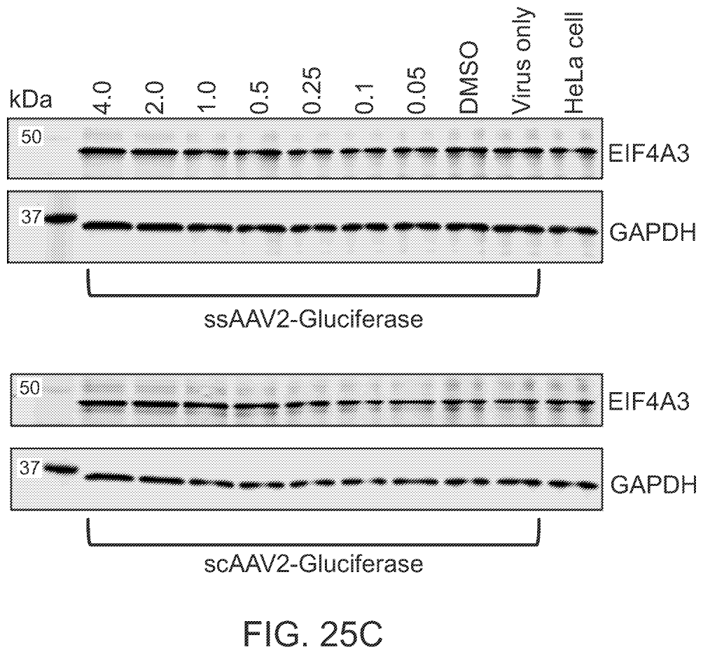

Zotatifin was found to not affect cell viability (FIGS. 23A and 23B). Zotatifin increases AAV transduction for various AAV serotypes (FIGS. 24A-F). Following pre-treatment of HeLa cells with zotatifin (FIG. 25A), ssAAV-Gaussia luciferace transduction increases but not scAAV-Gaussia luciferace (FIGS. 25B-C).

### Example 5. Protocol-siRNA Treatment and Transduction Efficiency

An example embodiment is shown in FIG. 7.
1. Day 0) Seed HEK 293/HeLa cells in 24 well plate.
2. Day 1) Transfection of 200 nM specific siRNAs including scRNA (negative control) with TransIT-X2 Dynamic Delivery System (mirus bio, Cat #MIR 60003) according to manufacturing instruction. Briefly, 1) Place 50 μl of Opti-MEMI Reduced-Serum Medium in a sterile tube; 2) Add 1 μl of a 100 μM siRNA stock solution (200 nM final concentration per well). Pipet gently to mix completely; 3) Add 1.5 μl of TransIT-X2. Pipet gently to mix completely; 4) Incubate at room temperature for 15 minutes to allow sufficient time for complexes to form; 5) Add the TransIT-X2:siRNA complexes drop-wise to different areas of the wells.
3. After 48 hrs of transfection, infect siRNA-treated cells with rAAV-Luciferase with MOI of 10,000.
4. After 24 hrs of infection, cells were harvested and then lysed with Reporter Lysis Buffer (Promega). Transduction efficiency was measured by Biotek Synergy HT Multi-Mode Microplate Reader.

### Example 6. Protocol-Chemical Treatment and Transduction Efficiency

An example embodiment is shown in FIG. 24A.
1. Day 0) Seed HeLa cells in 24 well plate.
2. Day 1) Treat cells with zotatifin (Cat #HY-112163, MCE MedChemExpress) using different doses (4 μM, 2 μM, 1 μM, 0.5 μM, 0.2 μM, 0.1 μM, 0.05 μM and DMSO as a control).
3. After 6 hrs of inhibitor treatment, infect cells with rAAV-Luciferase with MOI of 10,000.
4. After 24 hrs of infection, cells were harvested and then lysed with Reporter Lysis Buffer (Promega). Transduction efficiency was measured by Biotek Synergy HT Multi-Mode Microplate Reader.

In summary, mass spectroscopy analysis identified 77ea host proteins as candidates involved in AAV transduction. Ilea host proteins were evaluated and found to increase AAV transduction by siRNA screening (see Schreiber, C. A. et al., PLOS pathogens, 2015, 11(8), p.e1005082, which is incorporated herein by reference in its entirety). Knock-down of eIF4A3 does not play a role in attachment and internationalization of AAV. Knock-down of eIF4A3 slightly decreased nuclear entry and uncoated vector genome of AAV. eIF4A3 may affect the conversion from single strand to double strand DNA. eIF4A3 silencing increased AAV transgene expression from AAV. Both eIF4A3-IN-1 and zotatifin increased single strand AAV transduction.

### Example 7. Enhancement of AAV-Mediated Therapeutic Delivery of miRNAs

rAAV vectors will be constructed to evaluate the therapeutic delivery of miR-206 to breast cancer cells. MiR-206 functions as a tumor suppressor, and miR-206 levels in breast cancer cells when compared to normal breast cells are dramatically down regulated (see Chaudhari, R. et al., Scientific reports, 2022, 12(1), p. 4713, which is incorporated herein by reference in its entirety). Mouse models of MCF-7 or MDA-MB-231 xenograft tumors will be treated with host protein inhibitor (eIF4A3-IN-1 and zotatifin). To allow facile assessment of target tissue transduction, the vectors will include enhanced green fluorescent protein (eGFP) driven by the ubiquitously expressed elongation factor 1 alpha (EFla) promoter (see Kota, J. et al. Cell, 2009, 137(6), p. 1005, which is incorporated herein by reference in its entirety). Moreover, since miRNAs are often embedded within introns of both protein-coding and noncoding primary transcripts, miR-206 will be cloned into the short intron which is part of the EF1α promoter unit, thus allowing simultaneous production of eGFP and miR-206 from a single transcript. Vector expression of both miR-206 and eGFP will be confirmed by transient transfection of HeLa cells. The vectors will be packaged with various AAV serotypes for in vivo delivery. Vector genomes (vg) per animal will be administered with a single tail-vein injection and the xenograft tumors will be harvested three weeks later for analysis of miRNA and eGFP expression.

It is expected that pre-treatment of tumor with host protein inhibitor enhances AAV-mediated delivery of miR-206, suppresses tumorigenesisin treated mice, reduces cancer cell proliferation, and induces tumor-specific apoptosis. It is expected that the treated mice will exhibit only small tumors or a complete absence of tumors.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments.

What is claimed is:

1. A pharmaceutical composition comprising a vector, a host protein inhibitor or a stereoisomer, tautomer or salt thereof, and a pharmaceutically acceptable carrier, wherein the host protein is eIF4A1, eIF4A2, eIF4A3, RAP1A, NIFK, SART1, PLS1, or SF3B6, and wherein the vector comprises a transgene operably linked to regulatory sequences which direct expression of a product from the transgene in a cell.

2. The composition of claim 1, wherein the host protein is eIF4A3.

3. The composition of claim 1 or 2, wherein the host protein inhibitor is 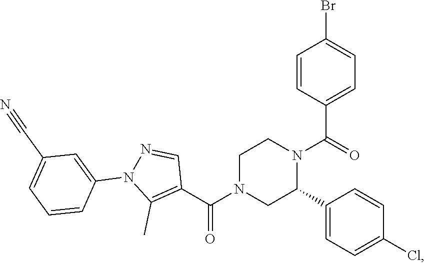 [SMILES: Cc1c(C(=O)N2CCN(C(=O)c3ccc(Br)cc3)[C@H](c3ccc(Cl)cc3)C2)cnn1-c1cccc(C#N)c1]
or a stereoisomer, tautomer or salt thereof.

4. The composition of claim 1 or 2, wherein the host protein inhibitor is 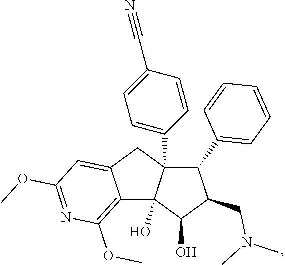
or a stereoisomer, tautomer or salt thereof.

5. The composition of any one of claims 1-4, wherein the vector is a viral vector or a non-viral vetor.

6. The composition of any one of claims 1-5, wherein the vector is a viral vector.

7. The composition of any one of claims 1-6, wherein the vector is an adeno-associated virus (AAV) vector.

8. The composition of any one of claims 1-7, wherein the vector is a self-complementary vector, a single-stranded vector, or a combination thereof.

9. The composition of any one of claims 1-8, wherein the vector is a single-stranded vector.

10. The composition of any one of claims 1-9, wherein the vector is an AAV1, AAV2, AAV3b, AAV5, or AAV9 vector.

11. A method of delivering a transgene to a cell in a subject, said method comprising administering to the subject a pharmaceutical composition comprising a vector and a host protein inhibitor or a stereoisomer, tautomer or salt thereof, wherein the host protein is eIF4A1, eIF4A2, eIF4A3, RAP1A, NIFK, SART1, PLS1, or SF3B6, and wherein the vector comprises the transgene operably linked to regulatory sequences which direct expression of a product from the transgene in the cell.

12. A method of increasing transduction efficiency of a vector in a cell, said method comprising contacting the cell with a host protein inhibitor or a stereoisomer, tautomer or salt thereof, wherein the host protein is eIF4A1, eIF4A2, eIF4A3, RAP1A, NIFK, SART1, PLS1, or SF3B6, and wherein the vector comprises a transgene operably linked to regulatory sequences which direct expression of a product from the transgene in the cell.

13. A method of treating a disease in a subject in need thereof, wherein the method comprises administering to the subject a pharmaceutical composition comprising a vector and a host protein inhibitor or a stereoisomer, tautomer or salt thereof, wherein the host protein is eIF4A1, eIF4A2, eIF4A3, RAP1A, NIFK, SART1, PLS1, or SF3B6, and wherein the vector comprises a transgene operably linked to regulatory sequences which direct expression of a product from the transgene in a cell.

14. The method of any one of claims 11-13, wherein the cell is a cancer cell.

15. The method of any one of claims 11-14, wherein the host protein is eIF4A3.

16. The method of any one of claims 11-15, wherein the host protein inhibitor is 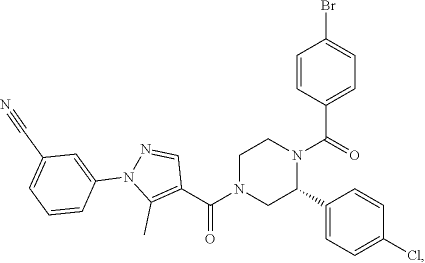 [SMILES: Cc1c(C(=O)N2CCN(C(=O)c3ccc(Br)cc3)[C@H](c3ccc(Cl)cc3)C2)cnn1-c1cccc(C#N)c1]
or a stereoisomer, tautomer or salt thereof.

17. The method of any one of claims 11-15, wherein the host protein inhibitor is 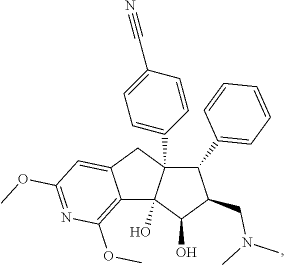
or a stereoisomer, tautomer or salt thereof.

18. The method of any one of claims 11-17, wherein the vector is a viral vector or a non-viral vector.

19. The method of any one of claims 11-18, wherein the vector is a viral vector.

20. The method of any one of claims 11-19, wherein the vector is an adeno-associated virus (AAV) vector.

21. The method of any one of claims 11-20, wherein the vector is a self-complementary vector, a single stranded vector, or a combination thereof.

22. The method of any one of claims 11-21, wherein the vector is a single stranded vector.

23. The method of any one of claims 11-22, wherein the vector is an AAV1, AAV2, AAV3b, AAV5, or AAV9 vector.

24. The composition of any one of claims 1-10, or the method of any one of claims 11-23, wherein the product of the transgene is a protein, peptide, or nucleic acid.

25. The composition of any one of claims 1-10, or the method of any one of claims 11-24, wherein the product of the transgene is an antibody or fragment thereof.

26. The composition of any one of claims 1-10, or the method of any one of claims 11-25, wherein the product is an siRNA, microRNA (miRNA), or antisense oligonucleotide (ASO).

27. The composition of any one of claims 1-10, or the method of any one of claims 11-26, wherein the product is an miRNA.

28. The method of claim 13, wherein the disease is selected from the group consisting of cancer, ischemia, diabetic retinopathy, macular degeneration, rheumatoid arthritis, psoriasis, HIV infection, sickle cell anemia, Alzheimer's disease, muscular dystrophy, neurodegenerative disease, vascular disease, cystic fibrosis, and stroke.

29. The method of claim 13 or 28, wherein the disease is cancer.

30. The method of claim 29, wherein the cancer comprises a solid tumor.

31. The method of claim 29 or 30, wherein the cancer is selected from one or more of breast cancer, bladder cancer, endometrial cancer, esophageal cancer, liver cancer, pancreatic cancer, lung cancer, cervical cancer, colon cancer, colorectal cancer, gastric cancer, kidney cancer, ovarian cancer, prostate cancer, testicular cancer, uterine cancer, a viral-induced cancer, melanoma or sarcoma.

32. The method of any of the claims 29-31, wherein the cancer is breast cancer or cervical cancer.

33. The method of any one of claims 29-32, wherein the cancer is breast cancer.

34. The method of any one of claims 29-32, wherein the cancer is cervical cancer.
